(19) United States
(12) Patent Application Publication
(10) Pub. No.: US 2004/0002159 A1
(43) Pub. Date: Jan. 1, 2004

(54) Methods for the production of chimeric adeno-associated virus (AAV) vectors, compositions of chimeric AAV vectors, and methods of use thereof

(76) Inventors: Weidong Xiao, Fort Washington, PA (US); Bernd Hauck, Philadelphia, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/408,695

(22) Filed: Apr. 7, 2003

Related U.S. Application Data
(60) Provisional application No. 60/370,288, filed on Apr. 5, 2002.

(51) Int. Cl. C12N 15/861; C12N 7/00; C12N 5/06

(52) U.S. Cl. 435/457; 435/235.1; 435/320.1; 435/325

(57) ABSTRACT

The present invention provides methods for making and using chimeric AAV particles having broad tissue tropism. The AAV particles may be used to deliver nucleic acid sequences encoding a desired protein wherein infection by chimeric AAV particles provides means to transduce cells with the nucleic acid sequences. Such gene transduction results in production of the desired protein in a cell, and thus establishes or restores the activity of the protein in the cell. The present invention also provides pharmaceutical compositions comprising such chimeric AAV particles for use in the therapeutic treatment of patients.

### CROSS REFERENCE TO RELATED APPLICATION

This application claims priority to U.S. provisional application 60/370,288 filed Apr. 5, 2002, the entire disclosure of which is incorporated by reference herein.

Pursuant to 35 U.S.C. §202(c), it is acknowledged that the U.S. Government has certain rights in the invention described herein, which was made in part with funds from the National Institute of Health, Grant No. R01HL069051.

### FIELD OF THE INVENTION

The present invention relates to methods for generating novel, chimeric adeno-associated virus (AAV) particles useful for transduction of genes into cells of diverse tissue origin. The present invention also provides methods for utilizing chimeric AAV vectors for therapeutic and/or prophylactic purposes.

### BACKGROUND OF THE INVENTION

Adeno-associated virus (AAV) is a single-stranded DNA parvovirus, which integrates into a host genome during the latent phase of infectivity. AAV2, for example, is a particular serotype of AAV which is endemic to the human population and frequently integrates site-specifically into human chromosome 19 q13.3. AAV is considered a dependovirus because it requires helper functions from either adenovirus or herpes-virus in order to replicate. In the absence of either of these helper viruses, AAV can infect cells, uncoat in the nucleus, and integrate its genome into the host cell chromosome, but can not replicate or produce new viral particles.

The genome of AAV is comprised of two terminal repeats which serve as origins of DNA replication and two functional regions. One functional region provides the rep genes which serve to regulate viral DNA replication and viral gene expression, whereas the second functional region provides the cap genes which encode the structural capsid proteins VP1, VP2 and VP3. The proteins encoded by both the rep and cap genes function in trans during productive AAV replication.

The five original AAV serotypes have been characterized and sequenced. Based on seroepidemological analyses, AAV1 and AAV4 were determined to be of non-human primate origin, whereas AAV2, AAV3 and AAV5 were classified as human in origin. In general, the above AAV serotypes share a high degree of homology in both the rep and cap regions, with the exception of AAV5, which is a more distantly related dependovirus. Despite the significant degree of homology maintained among the AAVs, each serotype appears to use a different set of cellular receptors and exhibit different tissue tropism. A sixth AAV serotype (AAV6) has recently been isolated and has been shown to be a recombinant product of AAV1 and AAV2. The exact nature of this hybrid virus is still unclear, but it is anticipated to possess features similar to those of AAV1 due to the high degree of identity between the respective cap proteins.

AAVs are considered ideal candidates for use as transducing vectors, in part, because none of the known serotypes have been linked to any human disease. This feature renders the AAVs distinct from autonomous parvoviruses, which can cause a variety of human disorders. Moreover, AAV virions are of interest as vectors for gene therapy because of their broad host range, excellent safety profile, and the duration of transgene expression in infected hosts. In general, AAV transducing vectors comprise sufficient cis-acting functions to replicate when adenovirus or herpesvirus helper functions are provided in trans. Recombinant AAV (rAAV) vectors have been constructed in a number of laboratories and have been used to transduce exogenous genes into cells of a variety of lineages. In these vectors, the AAV cap and/or rep genes are frequently deleted from the viral genome and replaced with a DNA segment of choice.

Recent developments have greatly improved AAV vector synthesis and facilitated the production of large quantities of wild type and helper virus rAAV vectors. Such developments involved the use of mini helper plasmids and the establishment of AAV producing cell lines which provide means to modulate AAV helper gene expression and helper size. The ability to generate large quantities of rAAV vectors has facilitated more extensive studies of these vectors in animals. Testing in a variety of animal tissues has confirmed that AAV-based vectors are useful tools for directed gene transfer experiments in vivo.

Most of the animal studies performed to date have utilized AAV vectors derived from AAV2, the first AAV serotype cloned and sequenced. The receptors and co-receptors identified for AAV2, include heparin sulfate proteoglycan (HSPG), fibroblast growth factor receptor (FGFR), and integrin αvβ5. The expression pattern of these receptors and co-receptors on cells derived from different tissue sources appears to dictate the susceptibility of a cell to AAV2 infection. Animal studies utilizing AAV2 have demonstrated that AAV2 vectors can deliver transgenes to a variety of cells including, muscle, liver and brain cells. The performance of AAV2-derived vectors in vivo also parallels the expression pattern of AAV2 receptors on host cells. A number of studies utilizing vectors based on other AAV serotypes have provided further evidence that different AAVs use different cellular receptors for entry, thus dictating their ability to infect cells of disparate tissue origin. For example, AAV5 has been reported to infect brain, liver and airway epithelial cells efficiently, whereas AAV1 infects muscle cells well and is therefore better suited for muscle-based gene transfer. The ability of AAV1 to infect muscle cells exceeds that of AAV2 by approximately 10-1000 fold, as determined by measuring levels of co-expressed transgenes having variable half lives. AAV2, however, is generally a better vector for liver-directed gene transfer.

### SUMMARY OF THE INVENTION

In accordance with the present invention, methods for making novel chimeric AAV vectors are provided. Such chimeric AAV vectors may be used to advantage in compositions for the prophylactic and/or therapeutic treatment of patients having a variety of human disorders.

In one aspect of the present invention, a method is provided for making chimeric adeno-associated (AAV) particles comprising capsid proteins from different AAV serotypes. An exemplary method comprises I) providing a vector construct wherein the vector construct encodes nucleic acid sequences encoding a desired polypeptide; ii) providing a helper virus construct wherein the helper virus provides accessory functions for production of viral particles; iii) combining at least two AAV helper constructs to produce an AAV helper construct mixture; and iv) introducing the vector construct, the helper virus construct, and the AAV helper construct mixture into a recipient cell to effect production of chimeric AAV particles capable of infecting diverse cell types. Chimeric AAV particles so produced are also within the scope of the present invention.

In other aspects of the present invention, a method is provided for making chimeric AAV particles, wherein a AAV helper construct mixture comprises two, three, four, five, or six different AAV helper constructs.

In a preferred embodiment, an AAV helper construct mixture comprises AAV1 and AAV2 helper constructs. Such an AAV helper construct mixture may be comprised of any ratio of AAV1 to AAV2 helper constructs. An AAV helper construct mixture may be comprised, for example, of 20 percent AAV1 helper construct and 80 percent AAV2 helper construct; 50 percent AAV1 helper construct and 50 percent AAV2 helper construct; or 80 percent AAV1 helper construct and 20 percent AAV2 helper construct.

A chimeric AAV particle or vector may comprise any nucleic acid sequence encoding a polypeptide. Such a nucleic acid sequence may be selected, for example, from the group of nucleic acid sequences provided in Table IV V.

In a particularly preferred embodiment, a chimeric AAV particle of the present invention comprises the nucleic acid sequence of Genbank No. J00136 (SEQ ID NO: 1, shown in FIG. 6), which encodes Factor IX polypeptide, Genbank No. K01396 (SEQ ID NO: 2, shown in FIG. 7), which encodes human α1-antitrypsin, or a protein encoded by any of the sequences shown in Table V.

In another aspect, compositions comprising the chimeric AAV particles of the present invention are provided.

In a particular aspect, compositions comprising chimeric AAV particles which encode Factor IX polypeptide, human α1-antitrypsin polypeptide, or any of the polypeptides encoded by the sequences shown in Table V are provided.

In yet another aspect of the invention, methods are provided for the treatment of a patient having a mutation in a gene, said method comprising delivery of a therapeutically effective amount of a composition comprising chimeric AAV particles of the present invention to cells of said patient. Such methods provide novel and improved methods for human gene therapy.

In one embodiment of the present invention, methods are provided for the treatment of a patient having a mutation in Factor IX, said method comprising delivery of a composition comprising chimeric AAV particles encoding Factor IX to cells of said patient.

In a preferred embodiment, chimeric AAV particles encoding Factor IX are delivered to muscle cells of a patient. In an alternative embodiment, chimeric AAV particles encoding Factor IX are delivered to liver cells of a patient.

In another embodiment of the present invention, methods are provided for the treatment of a patient having a mutation in human α1-antitrypsin, said method comprising delivery of a composition comprising chimeric AAV particles encoding human α1-antitrypsin to cells of said patient.

In a preferred embodiment, chimeric AAV particles encoding human α1-antitrypsin are delivered to muscle cells of a patient. In an alternative embodiment, chimeric AAV particles encoding human α1-antitrypsin are delivered to liver cells of a patient.

Aspects and embodiments of the present invention will now be illustrated, by way of example, with reference to the accompanying figures. Further aspects and embodiments will be apparent to those skilled in the art. All documents mentioned in this text are incorporated herein by reference.

### BRIEF DESCRIPTION OF THE DRAWINGS

FIG. 1 illustrates the strategy utilized to produce chimeric recombinant AAV vectors based on AAV1 and AAV2. AAV1 and AAV2 genomes and expressed proteins are differentially represented.

FIG. 2 demonstrates the production of chimeric vectors. The AAV1/AAV2 chimeric vectors were generated by co-transfection of pAAV.FIX, pAd and AAV helper into 3×10⁸ 293 cells. The ratios of AAV1 and AAV2 helper were 10:0, 9:1, 1:1, 1:9 and 0:10, respectively. The vectors were purified by Cesium Chloride (CsCl) gradient and those present in the density range of 1.38-1.42 g/ml were collected. Vectors were titered by quantitative PCR.

FIG. 3 shows the purification of chimeric vectors by heparin column chromatography. AAV1/AAV2 chimeric vectors were generated by co-transfection of pAAV.FIX, pAd and AAV helper into 3×10⁸ 293 cells. The ratios of AAV1 and AAV2 helper were 10:0, 9:1, 1:1, 1:9 and 0:10, respectively. The vectors were purified by heparin column chromatography and eluted over a NaCl gradient ranging from 200 mM to 1 M. Vectors were titered by quantitative PCR. The amount of vector in each elution fraction is shown. The Y-axis represents the ratio of the total amount of vector in each fraction relative to the amount of vector purified by CsCl gradient. The X-axis represents the salt concentration used for elution.

FIG. 4 shows the performance of chimeric AAV1/AAV2 vectors in muscle. 5×10¹⁰ particles were injected to the muscle of CD4 KO mice. The expression levels of human Factor IX at week 8 post-injection are shown. The following vectors were used: CsCl purified AAV1, AAV2, chimeric AAV1/AAV2 (produced following cotransfection with AAV1 and AAV2 at ratios of 9:1, 1:1 and 1:9).

FIG. 5 shows the performance of chimeric AAV vectors in liver. 5×10¹⁰ particles were injected into the tail vein of C57BL6 mice. The expression levels of human Factor IX at week 8 post-injection are shown. The following vectors were used: CsCl purified AAV1, AAV2, chimeric AAV1/AAV2 (produced following cotransfection with AAV1 and AAV2 at ratios of 9:1, 1:1 and 1:9).

FIG. 6 shows the polynucleotide sequence of Factor IX (SEQ ID NO: 1).

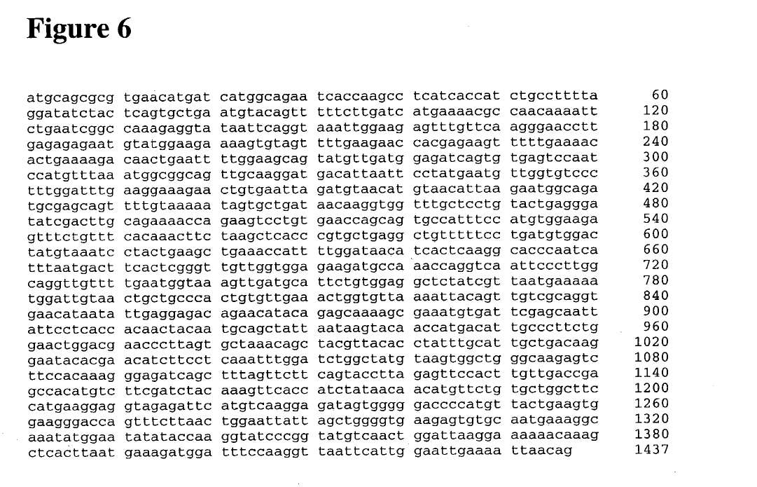

FIG. 7 shows the polynucleotide sequence of α1-antitrypsin (SEQ ID NO: 2).

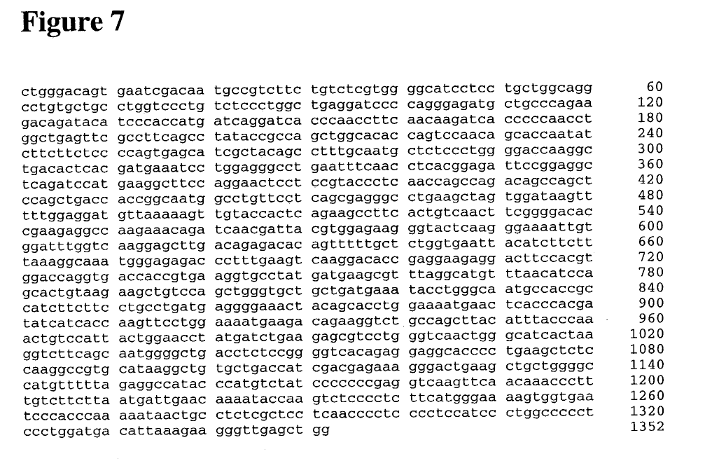

### DETAILED DESCRIPTION OF THE INVENTION

In accordance with the present invention, methods are provided for the production of novel, improved AAV-derived vectors. Also encompassed by the invention are the improved AAV-derived vectors so produced and methods of use thereof. The chimeric AAV particles of the invention have broad tissue tropism, and can thus be utilized to infect a wide range of target cell types in order to deliver therapeutically beneficial exogenous nucleic acid to a target cell.

I. Definitions

As used herein, the terms “gene transfer”, “gene delivery”, and “gene transduction” refer to methods or systems for reliably inserting a particular nucleotide sequence (e.g., DNA) into targeted cells.

As used herein, the terms “vector,” and “gene transfer vector” refer to any genetic element, such as a plasmid, phage, transposon, cosmid, chromosome, virus, virion, etc., which is capable of replication when associated with the proper control sequences and/or which can transfer nucleic acid sequences between cells. Thus, the term includes cloning and expression vectors, as well as viral vectors.

Gene transfer vectors may include transcription sequences such as polyadenylation sites, selectable markers or reporter genes, enhancer sequences, and other control sequences which allow for the induction of transcription. Such control sequences are described more fully below.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, as well as other sequences. Eukaryotic cells are generally known to utilize promoters (constitutive, inducible or tissue specific), enhancers, and termination and polyadenylation signals, although some elements may be deleted and other elements added without sacrificing the requisite expression.

As used herein, the terms “host” and “expression host” refer to organisms and/or cells which harbor an exogenous DNA sequence (e.g., via transfection), an expression vector or vehicle, as well as organisms and/or cells that are suitable for use in expressing a recombinant gene or protein. It is not intended that the present invention be limited to any particular type of cell or organism. Rather, the methods of the present invention are applicable to any organism and/or cell that is a suitable host.

As used herein, the terms “viral replicons” and “viral origins of replication” refer to viral DNA sequences that allow for the extrachromosomal replication of a vector in a host cell expressing the appropriate replication factors. In some embodiments, vectors which contain either the SV40 or polyoma virus origin of replication replicate to high copy number, while vectors which contain the replicons from bovine papillomavirus or Epstein-Barr virus replicate extrachromosomally at low copy number may be utilized in other embodiments.

As used herein, the term “adenoviral associated virus (AAV) vector” refers to a vector having functional or partly functional ITR sequences. As used herein, the term “ITR” refers to inverted terminal repeats. The ITR sequences may be derived from an adeno-associated virus serotype, including without limitation, AAV-1, AAV-2, AAV-3, AAV-4, AAV-5, and AAV-6. The ITRs, however, need not be the wild-type nucleotide sequences, and may be altered (e.g., by the insertion, deletion or substitution of nucleotides), so long as the sequences retain function to provide for functional rescue, replication and packaging. AAV vectors may have one or more of the AAV wild-type genes deleted in whole or part, preferably the rep and/or cap genes but retain functional flanking ITR sequences. Functional ITR sequences are necessary for the rescue, replication and packaging of the AAV virion. Thus, an “AAV vector” is defined herein to include at least those sequences required in cis for replication and packaging (e.g., functional ITRs) of the virus.

The terms “adeno-associated virus inverted terminal repeats” or “AAV ITRs” refer to the palindromic regions found at each end of the AAV genome which function together in cis as origins of DNA replication and as packaging signals for the virus. For use in some embodiments of the present invention, flanking AAV ITRs are positioned 5′ and 3′ of one or more selected heterologous nucleotide sequences. Optionally, the ITRs together with the rep coding region or the Rep expression products provide for the integration of the selected sequences into the genome of a target cell.

As used herein, the term “AAV rep coding region” refers to the region of the AAV genome which encodes the replication proteins Rep 78, Rep 68, Rep 52 and Rep 40. These Rep expression products have been shown to possess many functions, including recognition, binding and nicking of the AAV origin of DNA replication, DNA helicase activity and modulation of transcription from AAV (or other heterologous) promoters. The Rep expression products are collectively required for replicating the AAV genome. Muzyczka (Muzyczka, Curr. Top. Microbiol. Immunol., 158:97-129 [1992]) and Kotin (Kotin, Hum. Gene Ther., 5:793-801 [1994]) provide additional descriptions of the AAV rep coding region, as well as the cap coding region described below. Suitable homologues of the AAV rep coding region include the human herpesvirus 6 (HHV-6) rep gene which is also known to mediate AAV-2 DNA replication (Thomson el al., Virol., 204:304-311 [1994]).

As used herein, the term “AAV cap coding region” refers to the region of the AAV genome which encodes the capsid proteins VP1, VP2, and VP3, or functional homologues thereof. These cap expression products supply the packaging functions which are collectively required for packaging the viral genome.

As used herein, the term “AAV helper function” refers to AAV coding regions capable of being expressed in a host cell to complement AAV viral functions missing from the AAV vector. Typically, the AAV helper functions include the AAV rep coding region and the AAV cap coding region. An AAV helper construct as used herein, refers to a molecule that provides all or part of the elements necessary for AAV replication and packaging. Such AAV helper constructs may be a plasmid, virus or genes integrated into cell lines. It may be provided as DNA, RNA, or protein. The elements do not have to be arranged co-linearly (i.e., in the same molecule). For example, rep78 and rep68 may be on different molecules. An “AAV helper construct” may be, for example, a vector containing AAV coding regions required to complement AAV viral functions missing from the AAV vector (e.g., the AAV rep coding region and the AAV cap coding region).

As used herein, the terms “accessory functions” and “accessory factors” refer to functions and factors that are required by AAV for replication, but are not provided by the AAV vector or AAV helper construct. Thus, these accessory functions and factors must be provided by the host cell, a virus (e.g., adenovirus or herpes simplex virus), or another expression vector that is co-expressed in the same cell. Generally, the E1, E2A, E4 and VA coding regions of adenovirus are used to supply the necessary accessory function required for AAV replication and packaging (Matsushita et al., Gene Therapy 5:938 [1998]).

As used herein, the term “wild type” (“wt”) refers to a gene or gene product which has the characteristics of that gene or gene product when isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product which displays modifications in sequence and or functional properties (i.e., altered characteristics) when compared to the wild-type gene or gene product. It is noted that naturally-occurring mutants may be isolated, which are identified by the acquisition of altered characteristics when compared to the wild-type gene or gene product.

As used herein, the term “AAV virion” or “AAV particle” refers to a complete virus unit, such as a “wild-type” (wt) AAV virus particle (comprising a linear, single-stranded AAV nucleic acid genome associated with at least one AAV capsid protein coat). In this regard, single-stranded AAV nucleic acid molecules of either complementary sense (e.g., “sense” or “antisense” strands), can be packaged into any one AAV virion and both strands are equally infectious.

As used herein, the terms “recombinant AAV virion,” and “rAAV virion” refer to an infectious viral particle containing a heterologous DNA molecule of interest (e.g., Factor IX, human α1-antitrypsin, or a sequence shown in Table V) which is flanked on both sides by AAV ITRs. In some embodiments of the present invention, an rAAV virion is produced in a suitable host cell which contains an AAV vector, AAV helper functions and accessory functions. In this manner, the host cell is rendered capable of encoding AAV polypeptides that are required for packaging the AAV vector containing a recombinant nucleotide sequence of interest, such as Factor IX, human α1-antitrypsin, or a portion or functional fragment of Factor IX, or human α1-antitrypsin, into recombinant virion particles for subsequent gene delivery to patients suffering from hemophilia or α1-antitrypsin deficiency.

Hemophilia is a genetically inherited bleeding disorder which is caused by a deficiency of either blood clotting Factor VIII (hemophilia A) or IX (hemophilia B). As a result of impaired blood clotting function, hemophiliacs suffer from prolonged bleeding which can be life threatening and often leads to chronic disabilities (Lynch, 1999, Curr Opin Mol Ther 1:493-499).

Blood clotting or coagulation results from the concerted activities of a cascade of proteins. The Classic Cascade is composed of two basic parts, an intrinsic pathway and an extrinsic pathway. The intrinsic pathway is mediated through physical chemical activation and the extrinsic pathway is activated by tissue factor released from damaged cells. Both pathways are thought to be activated simultaneously to initiate and sustain clot formation.

The intrinsic pathway begins with trauma to a blood vessel, which results in the exposure of blood components to collagen in the damaged vascular wall. In response to such stimuli, Factor XII (also known as Hageman Factor) is converted from its inactive form (zymogen) to activated Factor XIIa. Platelets are also concomitantly activated as a consequence of trauma to blood vessels. Activated Factor XIIa is a protease which enzymatically activates Factor XI to become Factor XIa in a reaction which requires the presence of High Molecular Weight Kininogen and Prekallekrein. Activated Factor XI is also a protease, which functions to convert Factor IX (FIX) to the active protease Factor IXa, which is a Vitamin K-dependent serine protease that catalyzes the activation of Factor X to become Xa. Activation of Factor X is also greatly accelerated by Factor VIIIa. Activated Factor X functions as a protease which converts the inactive molecule prothrombin to the active thrombin in a process that requires the presence of Factor Va. Thrombin then cleaves fibrinogen to fibrin, which polymerizes to form fibrin strands.

Activation of the extrinsic pathway is initiated by trauma to vascular walls or extravascular tissue. Non-vascular tissue cells contain an integral membrane protein called tissue factor. Damage to the vessel wall or extravascular tissue exposes the plasma components to tissue factor. Thus, the circulating plasma protein Factor VII is exposed and binds to tissue factor to form a complex. As a consequence of this complex formation, Factor VII is activated to become Factor VIIa. In the presence of Ca++ and phospholipids, this complex activates Factor X to become Factor Xa. Once Factor Xa is generated, the remainder of the cascade is similar to the intrinsic pathway.

Thrombin is a powerful procoagulant which catalyzes the further conversion of Factors V and VIII to their activated forms through a positive feedback mechanism and converts more prothrombin to thrombin. In this manner, thrombin is able to accelerate the entire cascade once generated, resulting in the formation of large amounts of fibrin which are important for the formation of blood clots. Blood clots physically and biochemically prevent further bleeding at the site of an injury.

Activation through Factor VIIa/tissue factor occurs early in the course of fibrin clot formation, whereas activation by Factor XIa appears to be important for maintaining the integrity of the clot over time. In general, coagulation proteases are activated on a phospholipid surface in the presence of a protein cofactor. Recent evidence suggests that Factor XIa-mediated activation of Factor IX occurs on the surface of activated platelets in a biochemical reaction that differs significantly from other protease-substrate interactions on the platelet surface (Gailani, 2000, Trends Cardiovasc Med 10:198-204).

α1-antitrypsin deficiency is a genetically inherited disorder which occurs as a result of point mutations in the α1-antitrypsin gene which impair the protein's migration. α1-antitrypsin deficient homozygotes are predisposed to liver disease and pulmonary disorders (Lomas et al., (2002) J Clin Invest., 110(11):1585-1590.)

α1-antitrypsin is a serine proteinase inhibitor (serpin), and is the principle blood borne inhibitor of destructive neutrophil proteases. This glycoprotein is produced by the liver, secreted into serum, and travels to the lungs, where it protects elastin fibers and other connective tissue from degradation by neutrophil elastase (Song et al., (1998) PNAS 95:14384-14388.) Decreased levels of α1-antitrypsin in the lungs can produce pulmonary emphysema, due to uncontrolled degradation of connective tissue. Further, defective α1-antitrypsin can produce liver disease which occurs as a result of inclusions caused by accumulation of the defective glycoprotein in the rough ER of hepatic cells. (Lomas et al., supra.)

As used herein, the term “transfection” refers to the uptake of foreign DNA by a cell, and a cell has been “transfected” when exogenous DNA has been introduced inside the cell membrane. A number of transfection techniques are generally known in the art (See e.g., Graham et al., Virol., 52:456 [1973); Sambrook et al., Molecular Cloning, a Laboratory Manual, Cold Spring Harbor Laboratories, New York [1989]; Davis et al., Basic Methods in Molecular Biology, Elsevier, [1986]; and Chu et al., Gene 13:197 [1981]. Such techniques may be used to introduce one or more exogenous DNA moieties, such as a gene transfer vector and other nucleic acid molecules, into suitable recipient cells.

As used herein, the terms “stable transfection” and “stably transfected” refers to the introduction and integration of foreign DNA into the genome of the transfected cell. The term “stable transfectant” refers to a cell which has stably integrated foreign DNA into the genomic DNA.

As used herein, the term “transient transfection” or “transiently transfected” refers to the introduction of foreign DNA into a cell wherein the foreign DNA fails to integrate into the genome of the transfected cell and is maintained as an episome. During this time the foreign DNA is subject to the regulatory controls that govern the expression of endogenous genes in the chromosomes. The term “transient transfectant” refers to cells which have taken up foreign DNA but have failed to integrate this DNA. As used herein, the term “transduction” denotes the delivery of a DNA molecule to a recipient cell either in vivo or in vitro, via a replication-defective viral vector, such as via a recombinant AAV virion.

As used herein, the term “recipient cell” refers to a cell which has been transfected or transduced, or is capable of being transfected or transduced, by a nucleic acid construct or vector bearing a selected nucleotide sequence of interest (e.g., Factor IX). The term includes the progeny of the parent cell, whether or not the progeny are identical in morphology or in genetic make-up to the original parent, so long as the selected nucleotide sequence is present.

The term “heterologous” as it relates to nucleic acid sequences such as coding sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct can include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transfected with a construct which is not normally present in the cell would be considered heterologous for purposes of this invention. Allelic variation or naturally occurring mutational events do not give rise to heterologous DNA, as used herein.

As used herein, “coding sequence” or a sequence which “encodes” a particular protein, is a nucleic acid sequence which is transcribed (in the case of DNA) and translated (in the case of mRNA) into a polypeptide in vitro or in vivo, when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

As used herein, the term “nucleic acid” sequence refers to a DNA or RNA sequence. Nucleic acids can, for example, be single or double stranded. The term includes sequences such as any of the known base analogues of DNA and RNA.

As used herein, the term “recombinant DNA molecule” refers to a DNA molecule which is comprised of segments of DNA joined together by means of molecular biological techniques.

As used herein, the term “regulatory element” refers to a genetic element which controls the expression of nucleic acid sequences. For example, a promoter is a regulatory element which facilitates the initiation of transcription of an operably linked coding region. Other regulatory elements are splicing signals, polyadenylation signals, termination signals, etc. (defined infra).

The term DNA “control sequences” refers collectively to regulatory elements such as promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need be present so long as the selected coding sequence is capable of being replicated and is expressed appropriately in a recipient cell.

Transcriptional control signals in eukaryotes generally comprise “promoter” and “enhancer” elements. Promoters and enhancers consist of short arrays of DNA sequences that interact specifically with cellular proteins involved in transcription (Maniatis et al., Science 236:1237 [1987]). Promoter and enhancer elements have been isolated from a variety of eukaryotic sources including genes in yeast, insect and mammalian cells and viruses (analogous control sequences, i.e., promoters, are also found in prokaryotes). The selection of a particular promoter and enhancer depends on the recipient cell type. Some eukaryotic promoters and enhancers have a broad host range while others are functional in a limited subset of cell types (See e.g., Voss et al., Trends Biochem. Sci., 11:287 [1986]; and Maniatis et al., supra, for reviews). For example, the SV40 early gene enhancer is very active in a variety of cell types from many mammalian species and has been used to express proteins in a broad range of mammalian cells (Dijkema et al, EMBO J. 4:761 [1985]). Promoter and enhancer elements derived from the human elongation factor 1-alpha gene (Uetsuki et al., J. Biol. Chem., 264:5791 [1989]; Kim et al., Gene 91:217 [1990]; and Mizushima and Nagata, Nucl. Acids. Res., 18:5322 [1990]), the long terminal repeats of the Rous sarcoma virus (Gorman et al., Proc. Natl. Acad. Sci. U.S.A. 79:6777 [1982]), and the human cytomegalovirus (Boshart et al., Cell 41:521 [1985]) are also of utility for expression of proteins in diverse mammalian cell types. Promoters and enhancers can be found naturally, alone or together. For example, retroviral long terminal repeats comprise both promoter and enhancer elements. Generally promoters and enhancers act independently of the gene being transcribed or translated. Thus, the enhancer and promoter used can be “endogenous”, or “exogenous”, or “heterologous”, with respect to the gene to which they are operably linked. An “endogenous” enhancer/promoter is one which is naturally linked with a given gene in the genome. An “exogenous” or “heterologous” enhancer and promoter is one which is placed in juxtaposition to a gene by means of genetic manipulation (i.e., molecular biological techniques) such that transcription of that gene is directed by the linked enhancer/promoter.

As used herein, the term “tissue specific” refers to regulatory elements or control sequences, such as a promoter, enhancers, etc., wherein the expression of the nucleic acid sequence is substantially greater in a specific cell type(s) or tissue(s). In particularly preferred embodiments, the albumin promoter and the transthyretin promoter display increased expression of human Factor IX, human α1-antitrypsin, or a sequence shown in Table V, in hepatocytes, as compared to other cell types. It is not intended, however, that the present invention be limited to the albumin or transthyretin promoters or to hepatic-specific expression, as other tissue specific regulatory elements, or regulatory elements that display altered gene expression patterns, are encompassed within the invention. The use of tissue specific promoters that drive expression in muscle cells (e.g., the human muscle creatine kinase promoter) may be of particular utility in the present invention. Such promoters have been shown to avoid complications which arise from unwanted immune responses generated against the protein transduced via gene therapy (Weeratna et al., 2001, Gene Ther 8: 1872-1878).

The presence of “splicing signals” on an expression vector often results in higher levels of expression of the recombinant transcript. Splicing signals mediate the removal of introns from the primary RNA transcript and consist of a splice donor and acceptor site (Sambrook et al., Molecular Cloning: A Laboratory Manual, 2nd ed., Cold Spring Harbor Laboratory Press, New York [1989], pp. 16.7-16.8). A commonly used splice donor and acceptor site is the splice junction from the 16S RNA of SV40.

Transcription termination signals are generally found downstream of a polyadenylation signal and are a few hundred nucleotides in length. The term “poly A site” or “poly A sequence” as used herein denotes a DNA sequence which directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be “heterologous” or “endogenous.” An endogenous poly A signal is one that is found naturally at the 3′ end of the coding region of a given gene in the genome. A heterologous poly A signal is one which has been isolated from one gene and operably linked to the 3′ end of another gene. A commonly used heterologous poly A signal is the SV40 poly A signal. The SV40 poly A signal is contained on a 237 bp BamHI/BclI restriction fragment and directs both termination and polyadenylation (Sambrook et al., supra, at 16.6-16.7).

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

The phrase “consisting essentially of” when referring to a particular nucleotide or amino acid means a sequence having the properties of a given SEQ ID NO:. For example, when used in reference to an amino acid sequence, the phrase includes the sequence per se and molecular modifications that would not affect the basic and novel characteristics of the sequence.

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found as components of a mixture of numerous other mRNAs which encode a multitude of proteins in a cell. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of contaminants from a sample. For example, antibodies may be purified by removal of contaminating non-immunoglobulin proteins; they may also be purified by the removal of immunoglobulin that does not bind the antigen of interest. The removal of non-immunoglobulin proteins and/or the removal of immunoglobulins that do not bind the antigen of interest results in an increase in the percent of desired antigen-reactive immunoglobulins in the sample. In another example, recombinant polypeptides can be expressed in bacterial host cells and the polypeptides purified by the removal of host cell proteins; the percent of recombinant polypeptides is thereby increased in the sample.

As used herein, the term “hybrid protein” refers to two or more coding sequences obtained from different genes, that have been cloned together and that, after translation, are translated as a single polypeptide sequence. The coding sequences may be derived from different genes or portions thereof, which were obtained from the same species or from different species.

The term “tag,” “tag sequence” or “protein tag” refers to a chemical moiety, either a nucleotide, oligonucleotide, polynucleotide or an amino acid, peptide or protein or other chemical, that when added to another sequence, provides additional utility or confers useful properties, particularly in the detection or isolation of that sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose binding domain, calmodulin binding protein, maltose binding protein, chitin binding domain, glutathione S-transferase, and the like) may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin reagents, and the like. Numerous other tag moieties are known to, and can be envisioned by the trained artisan, and are contemplated to be within the scope of this definition.

The term “functional” as used herein implies that the nucleic or amino acid sequence is functional for the recited assay or purpose.

A “composition comprising a given polynucleotide sequence” as used herein refers broadly to any composition containing the given polynucleotide sequence. The composition can comprise an aqueous solution.

As used herein, the term “at risk” is used with regard to individuals who are at risk for experiencing episodes characterized by the manifestation of severe symptoms of a disease or disorder. In a particularly preferred embodiment, such “at risk” individuals can be patients with mild, moderate, or severe hemophilia, who are prone to hemorrhagic episodes.

As used herein, the term “subject” refers to any animal (i.e., vertebrates and invertebrates), while the term “vertebrate subject” refers to any member of the phylum Chordata. It is intended that the term encompass any member of this phylum, including, but not limited to humans and other primates, rodents (e.g., mice, rats, and guinea pigs), lagomorphs (e.g., rabbits), bovines (e.g, cattle), ovines (e.g., sheep), caprines (e.g., goats), porcines (e.g., swine), equines (e.g., horses), canines (e.g., dogs), felines (e.g., cats), domestic fowl (e.g., chickens, turkeys, ducks, geese, other gallinaceous birds, etc.), feral or wild animals, ungulates (e.g., deer), bear, and fish. It is not intended that the term be limited to a particular age or gender. Thus, adult and newborn subjects, as well as fetuses, whether male or female, are encompassed by the term.

As defined herein, an “immune response” signifies any reaction produced by an antigen, such as a viral antigen, in a host having a functioning immune system. Immune responses may be either humoral in nature, that is, involve production of immunoglobulins or antibodies, or cellular in nature, involving various types of B and T lymphocytes, dendritic cells, macrophages, antigen presenting cells and the like, or both. Immune responses may also involve the production or elaboration of various effector molecules such as cytokines, lymphokines and the like. Immune responses may be measured both in in vitro and in various cellular or animal systems. Such immune responses may be important in protecting the host from disease and may be used prophylactically and therapeutically.

As defined herein, a “therapeutically effective amount” or “therapeutic effective dose” is an amount or dose of AAV particles or virions capable of producing sufficient amounts of a desired protein to restore the activity of the protein, thus providing a palliative tool for clinical intervention. A therapeutically effective amount or dose of transfected AAV particles which confers expression of human Factor IX, for example, to a hemophiliac patient will decrease the time it takes for a subject's blood to clot. Additionally, a therapeutically effective amount or dose of transfected AAV particles which confer expression of human. α1-antitrypsin to a patient with an α1-antitrypsin deficiency will inhibit proteolytic damage from destructive neutrophil proteases.

The development of chimeric AAV particles as described herein facilitates the development of pharmaceutical compositions useful for the treatment of a variety of disorders, including diseases associated with genetic defects. An exemplary list of such disorders is provided in Table IV V. As defined herein, a “pharmaceutical composition” may comprise, in addition to at least one chimeric AAV particle, a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal, intrahepatic routes.

As used herein, the term “chimeric virus” refers to a virus that displays non-homogeneous capsid proteins (e.g., capsid proteins being derived from at least two different serotypes) in virions.

As used herein, the term “hybrid virus” refers to a virus which is comprised of nucleic acid sequences derived from a portion of one virus which has recombined with nucleic acid sequences derived from another virus. Such hybrid viruses are homogeneous in terms of composition.

As used herein, the term “gene therapy” refers to a method of treating a patient wherein polypeptides or nucleic acid sequences are transferred into cells of a patient such that activity and/or the expression of a particular molecule is restored.

II. Preparation of AAV Chimeric Vectors and Methods of Use Thereof

The present invention provides methods for the production of novel chimeric AAV vectors that infect a much broader range of cell types than previously described AAV vectors. Methods for the generation of chimeric AAV1/AAV2 vectors, for example, which exhibit useful tropic features of both AAV1 and AAV2 are provided. Chimeric AAV1/AAV2 vectors were generated by virtue of the extraordinarily high degree of homology maintained between the AAV1 and AAV2 genomes which facilitates the indiscriminate packaging of both the AAV1 and AAV2 cap proteins into individual mature virions. Mature virions having AAV1 and AAV2 cap proteins on their surface are useful tools for directed gene transduction because they exhibit features of both AAVs and can infect both liver and muscle cells efficiently.

Generally, AAV vectors are generated using a three-component system which includes a vector construct, a helper virus construct and an AAV helper construct. A vector construct comprises a plasmid which provides the cis signal from AAV for replication and packaging. A helper virus construct provides essential helper functions from adenovirus including: E1a, E1b, E2a, E4, and VA RNA. An AAV helper construct provides the rep and cap gene products to complement the replication and packaging processes. In traditional AAV production systems, only one AAV helper construct is used.

The present invention provides methods for the generation of novel chimeric rAAV particles which comprise both AAV1 and AAV2 capsid proteins. Such chimeric rAAV particles can be generated utilizing a mixture of AAV1 and AAV2 helper constructs. The AAV1 helper construct, for example, can comprise the AAV2 rep gene and AAV1 cap gene, whereas the AAV2 helper construct can comprise the AAV2 rep gene and the AAV2 cap gene. Since the AAV1 and AAV2 cap genes are more than 80% identical in amino acid sequence, the AAV packaging machinery can not distinguish between the two expressed capsid proteins and consequently produces chimeric virions comprised of both AAV1 and AAV2 capsid proteins.

The AAV1/AAV2 vectors of the present invention are classified as chimeric particles (also referred to herein as viroids or vectors), which are distinct from hybrid vectors such as AAV6. AAV6, for example, is comprised of nucleic acid sequences derived from a portion of AAV1 which has recombined with AAV2 nucleic acid-sequences. Overall, AAV6 virions remain homogeneous in terms of composition. In contrast, for the AAV1/AAV2 chimeric vectors, each virion has six different kinds of capsid proteins, including: AAV1 derived VP1, VP2, and VP3; and AAV2 derived VP1, VP2 and VP3. Since each cell is usually transfected with high copy numbers of AAV helper, it is estimated that more than a million copies of capsid proteins can be expressed in such a cell. From a statistical point of view, packaged virions produced from such cells in the late stage of AAV replication and packaging, when a vast pool of capsid proteins is available, would be nearly uniform in composition with regard to the AAV1 and AAV2 capsid proteins.

Nucleic acid sequences encoding a desired protein may be incorporated into the chimeric AAV vectors of the present invention to provide means to transfer the desired protein into cells. Chimeric AAV vectors comprising nucleic acid sequences encoding the desired protein may be used to infect cells having a genetic defect. Introduction of the nucleic acid encoding the wild type protein corrects the genetic defect by restoring expression of the protein. Accordingly, the present invention encompasses the treatment of cells having genetic defects wherein the chimeric AAV vectors may be used to infect the cells in vitro, in vivo, or ex vivo. Such chimeric AAV vectors may, therefore, be used in the efficacious treatment of disorders characterized by genetic deficiencies, such as, but not limited to Adenosine deaminase (ADA) deficiency, cystic fibrosis (CF), galactosemia (classic), Phenylketonuria (PKU), sickle cell anemia, Tay Sachs disease, Thalassemia (alpha and beta), Alpha-1 antitrypsin (α-1AT) deficiency, Canavan disease, Gaucher disease, Niemann-Pick Disease, hemophilia A, hemophilia B, Lesch-Nyhan syndrome, Familial hypercholesterolemia, Huntington's disease (HD), and Neurofibromatosis (NF) type I. See Table V for a list of GenBank Accession Numbers for nucleic acid sequences specified in which mutations have been identified that are linked to the above diseases.

Table V comprises an exemplary list of nucleic acid sequences or genes which may be incorporated into the chimeric vectors of the present invention to produce reagents of utility in the treatment of the indicated diseases/disorders. One of skill in the art would appreciate that nucleic acid sequences encoding any polypeptide or polypeptide fragment may be incorporated into the chimeric vectors of the present invention using standard cloning methods well known to those of ordinary skill in the art.

Nucleic acid sequences encoding human Factor IX, for example, may be incorporated into the chimeric AAV vectors to provide therapeutic agents useful for the treatment of hemophilia B patients having a genetic defect human Factor IX. (Poon et al., 2002, Thromb Haemost, 87:431-5 ). See Example I. Relatedly, results from phase I clinical trials using standard AAV vectors comprising the transgene for human Factor IX for the treatment of human hemophilia B patients have been encouraging. The initial dosage of AAV vector comprising the transgene human Factor IX (AAV-hFIX) used in human clinical studies, however, did not result in the expression of therapeutically beneficial levels of Factor IX. Higher doses of AAV-hFIX, therefore, appeared to be essential to render the Factor IX therapy beneficial to the patients. The benefits of such a high dose regimen of standard AAV vectors, however, may be outweighed by potential side effects such as an augmented immune response mounted in reaction to the bolus of viral particles introduced. Thus, the improved AAV vectors of the present invention, which enhance transgene expression without eliciting a heightened immune response, provide superior tools with which to introduce transgenes into host organisms for the treatment of genetic and metabolic disorders.

Also, nucleic acid sequences encoding Alpha-1 antitrypsin (α-1AT) may be incorporated into the chimeric AAV vectors to provide therapeutic agents useful for the treatment of Alpha-1 antitrypsin deficient patients. Murine studies using AAV Alpha-1 antitrypsin vectors indicate that this type of therapy is efficient, stable, non-toxic, and relatively non-immunogenic. This research also provides evidence that increasing doses of rAVV into muscle can increase serum levels of a therapeutic protein, such as Alpha-1 antitrypsin. Accordingly, current research indicates that gene therapy is a promising method for preventing emphysema in Alpha-1 antitrypsin deficient patients (Song et al., (1998) PNAS 95:14384-14388.)

The chimeric AAV1/AAV2 particles of the present invention are novel AAV vectors which demonstrate expanded tissue tropism, and thus, provide improved vehicles for the transduction of transgenes into host organisms. Since the chimeric AAV1/AAV2 particles can infect liver and muscle cells efficiently, utilization of these vectors for the treatment of disorders related to genetic deficiencies of these tissues may be particularly efficacious. Moreover, since the chimeric AAV vectors described herein provide more effective vehicles for the transfer of desired genes, they may be administered to patients at lower doses than standard AAV vectors. Thus, the chimeric AAV particles of the present invention provide improved agents for the therapeutic treatment of patients by AAV-mediated gene transfer.

In addition to the improved tissue tropism exhibited by the AAV1/AAV2 chimeric vectors, these vectors also demonstrate advantages with respect to the ease with which they can be purified. The introduction of AAV2 capsid protein on the surface of the chimeric particles facilitates purification of AAV1/AAV2 via heparin column, chromatography. Since the purity of heparin column purified vectors far exceeds that of vectors purified by CsCl gradient, the chimeric AAV1/AAV2 vectors also possess improved features with regard to safety measures that require the administration of accurately quantitated, standardized doses of pure reagents. In a preferred embodiment, the initial mix of AAV1 and AAV2 helper plasmids should comprise at least 50% AAV2 helper plasmid to ensure that the AAV1/AAV2 chimeric virions produced will comprise sufficient numbers of AAV2 capsid proteins to confer binding to heparin.

Since each AAV serotype appears to have different tissue tropism and all AAV serotypes share a high degree of homology, the present invention encompasses additional chimeric vectors produced utilizing the methods of the present invention. Such novel chimeric vectors would exhibit the combined properties of the plurality of parental AAV vectors used to generate the chimeric AAV vector.

Table I is provided to illustrate different combinations of parental AAV vectors which may be used to generate chimeric AAV vectors of the present invention. A combination is defined in the art of mathematics as one of the different arrangements of a group of items where the order of the items does not matter. The number of combinations of a group of n objects taken r at a time is:

C(n, r)=n!/r (−r)!

The definition of n! (n factorial) is (n)(n−1)(−2) . . . (3)(2)(1).

See Data Interpretation & Probability, Chapter 2, Nancy Parham (http://www.omegamath.com/data.html).

TABLE I
shows combinations of parental AAV serotypes

| Doublets | Triplets | Quadruplets | Quintuplets | Sextuplets |
|---|---|---|---|---|
| 1, 2 | 1, 2, 3 | 1, 2, 3, 4 | 1, 2, 3, 4, 5 | 1, 2, 3, 4, 5, 6 |
| 1, 3 | 1, 2, 4 | 1, 2, 3, 5 | 1, 2, 3, 4, 6 | |
| 1, 4 | 1, 2, 5 | 1, 2, 3, 6 | 1, 3, 4, 5, 6 | |
| 1, 5 | 1, 2, 6 | 1, 3, 4, 5 | 2, 3, 4, 5, 6 | |
| 1, 6 | 2, 3, 4 | 1, 3, 4, 6 | 1, 2, 4, 5, 6 | |
| 2, 3 | 2, 3, 5 | 1, 4, 5, 6 | 1, 2, 3, 5, 6 | |
| 2, 4 | 2, 3, 6 | 2, 3, 4, 5 | | |
| 2, 5 | 3, 4, 5 | 2, 4, 5, 6 | | |
| 2, 6 | 3, 4, 6 | 3, 4, 5, 6 | | |
| 3, 4 | 4, 5, 6 | 2, 3, 5, 6 | | |
| 3, 5 | 1, 3, 4 | 2, 3, 4, 6 | | |
| 3, 6 | 1, 3, 5 | 1, 3, 5, 6 | | |
| 4, 5 | 1, 3, 6 | 1, 2, 4, 6 | | |
| 4, 6 | 2, 4, 5 | 1, 2, 4, 5 | | |
| 5, 6 | 2, 4, 6 | 1, 2, 5, 6 | | |
| | 3, 5, 6 | | | |
| | 1, 4, 5 | | | |
| | 1, 5, 6 | | | |
| | 2, 5, 6 | | | |
| | 1, 4, 6 | | | |

Although data derived from animal studies suggest that AAV-mediated transduction results in prolonged transgene expression, which may even be maintained for the lifetime of the animal (Snyder et al. 1997, Nat Genet 16:270-276; Snyder et al. 1999, Nat Med 5:64-70; Chao et al. 2001, Mol Ther 4:217-222), the duration of AAV-mediated transgene expression in humans is not known. Moreover, the duration of AAV-mediated transgene expression in humans may be dependent on variables, including, but not limited to: the transgene expressed, the serotype of the AAV particle, the mode of introduction into a patient, and a patient's condition, age, and gender.

In the event that transgene expression decreases over time in a patient treated with a chimeric AAV vector of the present invention, it may be necessary to readminister the transgene to the patient.

AAV chimeric vectors may also be produced from modified AAV parental vectors, which possess altered immunological properties. Epitope mapping of the AAV2 capsid protein, for example, has identified seven regions of the protein which contain immunogenic epitopes (Moskalenko et al. 2000, J Virol 74:1761-1766). A parental AAV2 which has been mutated at these immunogenic epitopes should thus be rendered non-immunogenic and would, therefore, be of utility as a component of the chimeric AAV vectors of the present invention. Chimeric AAV vectors comprising mutated “non-immunogenic” AAV2 capsid proteins may be used to advantage for primary and/or secondary treatment of patients. Moskalenko et al. (supra) also provide six peptides which potentially reconstitute a single neutralizing epitope and thus may be used in conjunction with the chimeric AAV vectors of the present invention to reduce or neutralize an immune response to an AAV chimeric vector comprising AAV2.

III. Preparation of AAV Particles

AAV vectors of the present invention may be constructed using known techniques to provide, as operably linked components, (a) control sequences including transcriptional initiation and termination regions and (b) nucleotide sequences encoding a desired protein (e.g., Factor IX, human α1-antitrypsin, or a protein listed in Table V) or a functional fragment thereof. The control sequences are selected to be functional in a targeted recipient cell or cell type. The resulting construct which contains the operatively linked components is bounded (5′ and 3′) with functional AAV ITR sequences.

The nucleotide sequences of AAV ITR regions are known (See e.g., Kotin, Hum. Gene Ther., 5:793-801 [1994]; Berns, “Parvoviridae and Their Replication” in Fields and Knipe (eds), Fundamental Virology, 2nd Edition, for the AAV-2 sequence). AAV ITRs used in the vectors of the invention need not have a wild-type nucleotide sequence, and may be altered (e.g., by the insertion, deletion or substitution of nucleotides). Additionally, AAV ITRs may be derived from any of several AAV serotypes, including without limitation, AAV-1, AAV-2, AAV-3, AAV-4, AAV-5, and AAV6. Furthermore, 5′ and 3′ ITRs which flank a selected nucleotide sequence in an AAV vector need not necessarily be identical or derived from the same AAV serotype or isolate, so long as they function as intended.

A. Control Sequences

Heterologous control sequences can be incorporated into the vectors. Useful heterologous control sequences generally include those derived from sequences encoding mammalian or viral genes. Examples include, but are not limited to, the SV40 early promoter, mouse mammary tumor virus LTR promoter, adenovirus major late promoter (Ad MLP), a herpes simplex virus (HSV) promoter, a cytomegalovirus (CMV) promoter such as the CMV immediate early promoter region (CMVIE), a Rous sarcoma virus (RSV) promoter, a human muscle creatine kinase promoter, synthetic promoters, hybrid promoters, and the like. Alternatively, sequences derived from nonviral genes, such as the murine metallothionein gene can also be used. Such promoter sequences are commercially available (e.g., from Stratagene).

It is contemplated that in some embodiments, tissue-specific expression of a particular protein may be desirable (e.g., expression of biologically active Factor IX or human α1-antitrypsin by hepatocytes). It is not intended, however, that expression of biologically active Factor IX or human α1-antitrypsin be limited to any particular cells or cell types. Since hepatocytes (i.e., liver cells) normally synthesize Factor IX and human α1-antitrypsin, it is contemplated that in a preferred embodiment, the compositions of the present invention may be administered to the liver.

In preferred embodiments, expression may be achieved by coupling nucleic acid sequences encoding Factor IX or human α1-antitrypsin with heterologous control sequences derived from genes that are specifically transcribed by a selected tissue type. A number of tissue-specific promoters have been described above which enable directed expression in selected tissue types. Control sequences used in AAV vectors may also be used to drive expression of selected nucleic acid sequences. Of note in this regard, Maio et al. (2000, Mol Ther 1:522-532) found that expression of Factor IX in mice is stabilized by the inclusion of a hepatic locus control region, an hFIX intron, and a portion of the hFIX 3′ untranslated region in the expression plasmid. The Maio et al. (supra) is incorporated herein by reference in its entirety.

B. Construction of AAV Factor IX or Human α1-antitrypsin Vectors

AAV vectors that contain a control sequence and a nucleotide sequence of interest, bounded by AAV ITRs (i.e., AAV vectors), can be constructed by directly inserting selected sequences into an AAV genome with the major AAV open reading frames (“ORFs”)excised. Other portions of the AAV genome can also be deleted, so long as a sufficient portion of the ITRs remain to allow for replication and packaging functions. These constructs can be designed using techniques well known in the art (See e.g., U.S. Pat. Nos. 5,173,414 and 5,139,941, all of which are herein incorporated by reference); International Publication Nos. WO 92/01070 and WO 93/03769; Lebkowski et al., Mol. Cell. Biol., 8:3988-3996 [1988]; Vincent et al., Vaccines 90 [Cold Spring Harbor Laboratory Press, 1990]; Carter, Curr. Opin. Biotechnol., 3:533-539 [1992]; Muzyczka, Curr. Top. Microbiol. Immunol., 158:97-129 [1992]; Kotin, Hum. Gene Ther., 5:793-801 [1994]; Shelling and Smith, Gene Ther., 1:165-169 [1994]; and Zhou et al., J. Exp. Med., 179:1867-1875 [1994]).

Alternatively, AAV ITRs can be excised from the viral genome or from an AAV vector containing the same and fused 5′ and 3′ of a selected nucleic acid construct that is present in another vector using standard ligation techniques, such as those described in Sambrook et al., supra. For example, ligations can be accomplished in 20 mM Tris-Cl (pH 7.5), 10 mM MgCl₂, 10 mM DTT, 33 μg/ml BSA, 10 mM-50 mM NaCl, and either 40 CM ATP, 0.01-0.02 (Weiss) units T4 DNA ligase at 0° C. (for “sticky end” ligation) or 1 mM ATP, 0.3-0.6 (Weiss) units T4 DNA ligase at 4° C. (for “blunt end” ligation). Intermolecular “sticky end” ligations are usually performed at 30-100 μg/ml total DNA concentrations (5-100 nM total end concentration). AAV vectors which contain ITRs have been described in (e.g., U.S. Pat. No. 5,139,941, herein incorporated by reference). In particular, several AAV vectors are described therein which are available from the American Type Culture Collection (“ATCC”) under Accession Numbers 53222, 53223, 53224, 53225 and 53226.

Recombinant proteins may also be expressed utilizing the methods of the present invention. The nucleic acid sequences encoding a protein of interest can be fused in frame, with respect to the translational reading frame, with nucleic acid sequences encoding tags or epitopes. Such tags and/or epitopes are used routinely by skilled practitioners to track protein expression and as means to purify recombinant proteins. For example, a recombinant protein comprising a histidine tag (6-8 histidine residues) may be purified by column chromatography over nickel-bound beads. Alternative tags may include, but are not limited to, the FLAG epitope, the hemagglutinin epitope, GST, and fluorescent tags, such as green fluorescent protein (GFP).

Moreover, it is not intended that the present invention be limited to any specific Factor IX or human α1-antitrypsin sequence. Many natural and recombinant forms of Factor IX and human α1-antitrypsin have been isolated and assayed both in vitro and in vivo, using a variety of different regulatory elements and control sequences. Therefore, any known DNA sequences coding for biologically active proteins such as Factor IX, human α1-antitrypsin, or a protein listen in Table V can be expressed, alone or in combination with at least one additional vector, using the AAV vectors and methods of the present invention.

Nucleic acid sequences coding for the above-described proteins may be obtained using recombinant methods, such as by screening cDNA and genomic libraries from cells expressing proteins or by deriving the sequence from a vector known to include the same. Furthermore, the desired sequence may be isolated directly from cells and tissues containing the same, using standard techniques, such as phenol extraction and PCR of cDNA or genomic DNA (See e.g., Sambrook et al., supra, for a description of techniques used to obtain and isolate DNA). Nucleotide sequences encoding a protein of interest may also be produced synthetically, rather than cloned. The complete sequence may be assembled from overlapping oligonucleotides prepared by standard methods and assembled into a complete coding sequence (See e.g., Edge, Nature 292:756 [1981]; Nambair et al., Science 223:1299 [1984]; and Jay et al., J. Biol. Chem., 259:6311 [1984]).

Although it is not intended that the present invention be limited to any particular methods for assessing the production of biologically active proteins such methods as immunoassays (e.g., ELISA) and biological activity assays (e.g., coagulation activity assays, or proteinase inhibition assays) are contemplated.

Furthermore, it is not intended that the present invention be limited to the transduction of human sequences encoding human proteins. Indeed, it is intended that the present invention encompass transduction of therapeutically beneficial sequences from animals other than humans, including but not limited to companion animals (e.g., canines, felines, and equines), livestock (e.g., bovines, caprines, and ovines), laboratory animals (e.g., rodents such as murines, as well as lagomorphs), and “exotic” animals (e.g., marine mammals, large cats, etc.).

IV. Virion Production

The following is an exemplary embodiment of the present invention wherein nucleic acid sequences encoding Factor IX or human α1-antitrypsin are incorporated into the AAV vector to generate rAAV virions comprising Factor IX or human α1-antitrypsin which can transduce cells. One of skill in the art would appreciate that nucleic acid sequences encoding other proteins of interest such as those listed in Table V may be incorporated into the chimeric AAV vectors of the present invention. Generation of Factor IX or human α1-antitrypsin expressing rAAV virions generally involves the steps of: (1) introducing an AAV vector containing the Factor IX or human α1-antitrypsin gene into a host cell; (2) introducing one or more helper viruses and/or accessory function vectors into the host cell; (3) introducing a mixture of AAV helper constructs into the host cell; and (4) culturing the host cell to produce rAAV virions.

The above-described vectors and constructs can be introduced into a cell using standard methodology known to those of skill in the art (e.g., transfection). A number of transfection techniques are generally known in the art (See e.g., Graham el al., Virol., 52:456 [1973], Sambrook et al. supra, Davis et al., supra, and Chu et al., Gene 13:197 [1981]). Particularly suitable transfection methods include calcium phosphate co-precipitation (Graham et al., Virol., 52:456-467 [1973]), direct micro-injection into cultured cells (Capecchi, Cell 22:479-488 [1980]), electroporation (Shigekawa et al., BioTechn., 6:742-751 [1988]), liposome-mediated gene transfer (Mannino et al., BioTechn., 6:682-690 [1988]), lipid-mediated transduction (Felgner et al., Proc. Natl. Acad. Sci. U.S.A. 84:7413-7417 [1987]), and nucleic acid delivery using high-velocity microprojectiles (Klein et al., Nature 327:70-73 [1987]).

For the purposes of the invention, suitable host cells for producing rAAV virions include microorganisms, yeast cells, insect cells, and mammalian cells, that may be, or have been, used as recipients of a heterologous DNA molecule. The term includes the progeny of the original cell which has been transfected. Thus, as indicated above, a “host cell” as used herein generally refers to a cell which has been transfected with an exogenous DNA sequence. Cells from the stable human cell line, 293 (ATCC Accession No. CRL1573) are preferred in the practice of the present invention. The 293 cell line is a human embryonic kidney cell line that has been transformed with adenovirus type-5 DNA fragments (Graham et al., J. Gen. Virol., 36:59 [1977]), and expresses the adenoviral E1a and E1b genes (Aiello et al., Virol., 94:460 [1979]). The 293 cell line is readily transfected, and thus provides a particularly useful system in which to produce rAAV virions.

Host cells containing the above-described AAV vectors must be rendered capable of providing AAV helper functions in order to replicate and encapsidate the nucleic acid sequences flanked by the AAV ITRs to produce rAAV virions. AAV helper functions are generally AAV-derived coding sequences which can be expressed to provide AAV gene products that, in turn, function in trans for productive AAV replication. AAV helper functions are used herein to complement necessary AAV functions that are missing from the AAV vectors. Thus, AAV helper functions include one or both of the major AAV ORFs, namely the rep and cap coding regions, or functional homologues thereof.

AAV helper functions are introduced into the host cell by transfecting the host cell with a mixture of AAV helper constructs either prior to, or concurrently with, the transfection of the AAV vector. AAV helper constructs are thus used to provide at least transient expression of AAV rep and/or cap genes to complement missing AAV functions that are necessary for productive AAV infection. AAV helper constructs lack AAV ITRs and can neither replicate nor package themselves.

Mixtures of AAV helper constructs can comprise constructs containing cap genes derived from at least two AAV serotypes. In a preferred embodiment, AAV helper constructs comprising AAV1 and AAV2 cap genes may be mixed in various ratios to produce a helper construct mix. Infection of a recipient cell with such a helper construct mix, in conjunction with a vector construct and helper virus construct (as described herein), may be used to produce chimeric AAV1/AAV2 particles. AAV1 and AAV2 vectors may be combined in a range of different ratios to produce mixtures which are capable of conferring serotype-specific properties to the chimeric AAV particles produced following infection. Such ratios may range from, for example, 9:1, 4:1, 1:1, 1:4, to 1:9 (with respect to the molar amount of AAV1 and AAV2) for mixtures comprising AAV1 and AAV2 helper constructs. In a particular embodiment, the ratio of AAV2 to AAV1 helper vectors in the mixture may be adjusted (e.g., one part AAV1 to nine parts AAV2) to enhance the likelihood that chimeric AAV1/AAV2 particles produced as described above will have features more distinctive of AAV2 than AAV1.

In other embodiments, AAV helper constructs comprising cap genes derived from at least two different AAV serotypes may be mixed in various ratios to produce a helper construct mix. Infection of a recipient cell with such a helper construct mix, in conjunction with a vector construct and helper virus construct (as described herein), may be used to produce chimeric AAV particles. AAV serotype vectors may be combined in a range of different ratios to produce mixtures which are capable of conferring serotype-specific properties to the chimeric AAV particles produced following infection. Such ratios may range from, for example, 9:1, 4:1, 1:1, 1:4, to 1:9 (with respect to the molar amount of one AAV serotype compared to a second AAV serotype) for mixtures comprising two AAV helper constructs. For mixtures comprising at least two different AAV serotypes (i.e., two, three, four, five, or six different AAV serotypes), one of skill in the art would vary the molar ratio of the different serotypes to produce a chimeric AAV vector having suitable properties for a particular application.

In preferred embodiments, these constructs are in the form of a vector, including, but not limited to, plasmids, phages, transposons, cosmids, viruses, or virions. A number of AAV helper constructs have been described, such as the commonly used plasmids pAAV/Ad and pIM29+45 which encode both Rep and Cap expression products (See e.g., Samulski et al., J. Virol., 63:3822-3828 [1989]; and McCarty et al., J. Virol., 65:2936-2945 [1991]). A number of other vectors have been described which encode Rep and/or Cap expression products (See e.g., U.S. Pat. No. 5,139,941, incorporated herein by reference).

Both AAV vectors and AAV helper constructs may be constructed to contain one or more optional selectable markers. Suitable markers include genes which confer antibiotic resistance or sensitivity to, impart color to, or change the antigenic characteristics of those cells which have been transfected with a nucleic acid construct containing the selectable marker when the cells are grown in an appropriate selective medium. Several selectable marker genes are useful in the practice of the present invention, including, without limitation, the gene encoding neomycin that allows selection in mammalian cells by conferring resistance to G418 (Sigma). Other suitable markers are known to those of skill in the art.

The host cell (or packaging cell) should also be rendered capable of providing non-AAV derived functions, or “accessory functions,” in order to produce rAAV virions. Accessory functions are non-AAV derived viral and/or cellular functions upon which AAV is dependent for its replication. Thus, accessory functions include at least those non-AAV proteins and RNAs that are required in AAV replication, including those involved in activation of AAV gene transcription, stage specific AAV mRNA splicing, AAV DNA replication, synthesis of rep and cap proteins and AAV capsid assembly. Viral-based accessory functions can be derived from any of the known helper viruses.

Accessory functions may be transfected into host cells using methods known to those of skill in the art. Commonly, accessory functions are provided by infection of the host cells with an unrelated helper virus. A number of suitable helper viruses are known, including adenoviruses; herpesviruses such as herpes simplex virus types 1 and 2; and vaccinia viruses. Alternatively, accessory functions may be provided using an accessory function vector. Accessory function vectors include nucleotide sequences that provide one or more accessory functions. An accessory function vector is capable of being introduced into a suitable host cell in order to support efficient AAV virion production in the host cell. Accessory function vectors may be in the form of a plasmid, phage, virus, transposon or cosmid. Accessory vectors may also be in the form of one or more linearized DNA or RNA fragments which, when associated with the appropriate control sequences and enzymes, may be transcribed or expressed in a host cell to provide accessory functions.

Nucleic acid sequences providing the accessory functions may be obtained from natural sources, such as from the genome of adenovirus, or constructed using recombinant or synthetic methods known in the art. In this regard, adenovirus-derived accessory functions have been widely studied, and a number of adenovirus genes involved in accessory functions have been identified and partially characterized (See e.g., Carter, “Adeno-Associated Virus Helper Functions,” in CRC Handbook of Parvoviruses, Vol. I (P. Tijssen, ed.) [1990], and Muzyczka, Curr. Top. Microbiol. Immun., 158:97-129 [1992]). Specifically, early adenoviral gene regions E1a, E2a, E4, VAI RNA and, possibly, E1b are thought to participate in the accessory process (Janik et al., Proc. Natl. Acad. Sci. U.S.A. 78:1925-1929 [1981]). Herpesvirus-derived accessory functions (See e.g., Young et al., Prog. Med. Virol., 25:113 [1979])and vaccinia virus-derived accessory functions have also been described (See e.g., Carter, supra., and Schlehofer et al., Virol., 152:110-117 [1986]).

As a consequence of the infection of the host cell with a helper virus, or transfection of the host cell with an accessory function vector, accessory functions are expressed which transactivate the AAV helper constructs to produce AAV Rep and/or Cap proteins. The Rep expression products direct excision of the recombinant DNA (including, for example, nucleic acid sequences encoding Factor IX, human α1-antitrypsin, a protein shown in Table V, or a functional fragment thereof) from the AAV vector. The Rep proteins also serve to duplicate the AAV genome. The expressed Cap proteins assemble into capsids, and the recombinant AAV genome is packaged into the capsids. Thus, productive AAV replication ensues and the DNA is packaged into rAAV virions.

Following recombinant AAV replication, chimeric rAAV virions may be purified from the host cell using a variety of conventional purification methods, such as CsCl gradients. As described hereinabove, chimeric rAAV virions having AAV2 capsid proteins displayed on their surface may be purified via a heparin chromatography column. Further, if helper virus infection is employed to express the accessory functions, residual helper virus may be inactivated, using known methods. For example, adenovirus can be inactivated by heating to temperatures of approximately 60° C. for approximately 20 minutes or more, as appropriate. This treatment selectively inactivates the helper adenovirus which is heat labile, while preserving the rAAV which is heat stable.

V. Pharmaceutical Compositions

The resulting chimeric rAAV virions may be incorporated into pharmaceutical compositions that may be delivered to a subject, so as to allow production of a biologically active protein (e.g., Factor IX or human α1-antitrypsin). In a particular embodiment of the present invention, pharmaceutical compositions comprising sufficient genetic material to enable a recipient to produce a therapeutically effective amount of Factor IX which can reduce, stop and/or prevent hemorrhage in the subject are provided. Alternatively, pharmaceutical compositions comprising sufficient genetic material to enable a recipient to produce a therapeutically effective amount of human α1-antitrypsin which can inhibit proteolytic damage from destructive neutrophil proteases are provided. The compositions may be administered alone or in combination with at least one other agent, such as a stabilizing compound, which may be administered in any sterile, biocompatible pharmaceutical carrier, including, but not limited to, saline, buffered saline, dextrose, and water. The compositions may be administered to a patient alone, or in combination with other agents, clotting factors or factor precursors, drugs or hormones. In preferred embodiments, the pharmaceutical compositions also contain a pharmaceutically acceptable excipient. Such excipients include any pharmaceutical agent that does not itself induce an immune response harmful to the individual receiving the composition, and which may be administered without undue toxicity. Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. An exemplary pharmaceutical preparation may be a lyophilized powder which may contain any or all of the following: 1-50 mM histidine, 0.1%-2% sucrose, and 2-7% mannitol, at a pH range of 4.5 to 5.5, that is combined with buffer prior to use.)A thorough discussion of pharmaceutically acceptable excipients is available in Remington's Pharmaceutical Sciences (Mack Pub. Co., 18th Edition, Easton, Pa. [1990]).

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art. The pharmaceutical compositions of the present invention may be manufactured in any manner known in the art (e.g., by means of conventional mixing, dissolving, granulating, dragee-making, levigating, emulsifying, encapsulating, entrapping, or lyophilizing processes).

After pharmaceutical compositions have been prepared, they may be placed in an appropriate container and labeled for treatment. For administration of Factor IX-containing or human α1-antitrypsin-containing vectors, such labeling would include amount, frequency, and method of administration.

Pharmaceutical compositions suitable for use in the invention include compositions wherein the active ingredients are contained in an effective amount to achieve the intended therapeutic purpose. Determining a therapeutically effective dose is well within the capability of those skilled in the art using the techniques taught in the present invention. ELISAs (Herzog et al. 1999. Nature Med 5: 56-63) are a preferred method for measuring circulating serum levels of Factor IX or human α1-antitrypsin following gene therapy, although other techniques known in the art may also be used. Therapeutic doses of Factor IX (FIX) will depend on, among other factors, the age and general condition of the subject, the severity of hemophilia, and the strength of the control sequences. Therapeutic doses of human α1-antitrypsin (α-1AT) will depend on, among other factors, the age and general condition of the subject, the severity of the human α1-antitrypsin deficiency, and the strength of the control sequences. Thus, a therapeutically effective amount in humans will fall in a relatively broad range that may be determined based on the result of clinical trials and the response of individual patients to treatment.

The safety and efficacy of recombinant human factor IX (rFIX) has been evaluated in a 20-center international trial, which involved patients with severe or moderate (<5 IU/dL Factor IX activity) hemophilia B who had been previously treated with human plasma-derived Factor IX (pdFIX; Roth et al., 2001, Blood 98: 3600-3606). Participants received rFIX for pharmacokinetic studies, treatment of hemorrhage, or prophylaxis against hemorrhage, or surgical hemostasis. At baseline, an infusion of 50 IU/kg rFIX resulted in a mean FIX activity of 0.75 IU/dL per IU/kg, which corresponds to a recovery of 33.7%. The mean elimination half-life was 19.3 hours. These parameters as described above for recovery and elimination of FIX were demonstrated to be stable over the course of the two year study. Depending on the circumstances, patients received infusions comprising doses of rFIX ranging from ˜35 to 61 IU/kg (Roth et al., 2001, Blood 98: 3600-3606). Infusions were repeated as necessitated by the extent of the hemorrhage or potential for hemorrhage. For regular secondary prophylaxis regimens, for example, patients received infusions comprised of an average dose of 40.3 IU/kg (range 13-78 IU/kg) two to three times per week. For surgical prophylaxis, the dose was adjusted accordingly to counteract the degree of hemorrhage anticipated to result from the surgical procedure. Some patients required more than one dose per day to prevent or control hemorrhagic events.

Using computer simulations of FIX clearance, Bjorkman et al. (2001, Hemophilia 7:133-139) examined possible age-related changes in the disposition of FIX coagulant activity (FIX:C) after administration of rFIX. This study revealed that clearance and volume of distribution at steady state of FIX:C increased linearly with body weight of the patient. Thus, FIX:C remained fairly stable in adults, but varied during childhood and adolescence, which are developmental stages characterized by growth. The terminal half-life of FIX:C showed no correlation with age. In vivo recovery (in U/dL per U/kg) did, however, appear to correlate with age as evidenced by the tendency of the computer-predicted trough levels of exogenous FIX:C to increase during repeated doses of rFIX (50 U/kg). Conversely, doses (in U/kg) needed to maintain a 1 U/dL trough level showed little or no age dependence. Bjorkman et al. further demonstrated that considerable inter-individual variation existed in FIX:C and the doses of rFIX required to maintain therapeutically effective levels of FIX, thus emphasizing the need for individual dose titration. The authors of the study concluded that during prophylaxis, a 1 U/dL trough level of FIX can normally be maintained by dosing every two to three days.

VI. Administration

Chimeric AAV vectors of the present invention comprising nucleic acid sequences encoding a desired protein may be administered to a patient by a variety of means (see below) to achieve and maintain a prophylactically and/or therapeutically effective level of the particular protein. In one aspect of the invention, the particular protein encoded by a nucleic acid sequence may be chosen to supplement or restore the levels of the protein in a patient. An attending physician may identify a patient who would benefit from such intervention by providing a definitive diagnosis of the protein deficiency, which typically result from genetic and/or biochemical defects. See Table IV V.

One of skill in the art would also appreciate that the chimeric vectors of the present invention may also be used to advantage to supplement and/or restore expression of a particular protein for the purposes of therapeutic intervention in a patient who does not exhibit a genetic and/or biochemical defect. Such a patient may benefit from augmented levels of a particular protein (e.g., erythropoietin, cytokine, neuropeptide, hormone) to accelerate the rate of recovery following a medical procedure, such as, but not limited to surgery, chemotherapy, and/or radiation therapy. A clinician may also identify patients who, as a consequence of disease (e.g., infection) or impaired condition (e.g., elderly or weakened physically) would benefit from such intervention. One of skill in the art could readily determine specific protocols for using the chimeric AAV vectors of the present invention for the prophylactic and/or therapeutic treatment of a particular patient. Protocols for the generation of AAV vectors and administration to patients have been described in U.S. Pat. Nos. 6,335,011; 6,221,349; 6,211,163; 6,200,560; 6,027,931; 6,004,797; 6,001,650; 5,962,313; 5,952,221; 5,945,335; 5,858,351; 5,846,528; 5,843,742; and 5,622,856, which are incorporated herein by reference in their entirety.

Chimeric AAV vectors of the present invention comprising nucleic acid sequences encoding, for example, hFIX or α-1AT may be administered to a patient by a variety of means (see below) to achieve and maintain a therapeutically effective level of circulating hFIX of α-1AT. A therapeutically effective level of hFIX or α-1AT may be in the range of 0.1 to 10 U/dL. In a preferred embodiment, a therapeutically effective 1 U/dL trough of hFIX or α-1AT may be maintained by utilizing chimeric AAV vectors of the present invention which encode hFIX or α-1AT. It is intended that the dosage treatment and regimen used with the present invention will vary, depending upon the subject and the preparation used. Thus, the dosage treatment may be a single dose schedule or a multiple dose schedule. Moreover, the subject may be administered as many doses as appropriate to achieve or maintain the desired blood clotting time. Methods pertaining to the administration of standard AAV vectors to humans have been previously described by Kay et al. (2000, Nat Genet 24:257-261), the entire contents-of which is incorporated herein by reference. In certain circumstances when multiple vector administrations are necessary, it may be desirable to administer and express a protein of interest in a chimeric AAV vector which differs from the chimeric AAV vector initially administered.

Chimeric vectors of the present invention may be administered to a patient by any means known. Direct delivery of the pharmaceutical compositions in vivo may generally be accomplished via injection using a conventional syringe, although other delivery methods such as convection-enhanced delivery are envisioned (See e.g., U.S. Pat. No. 5,720,720, incorporated herein by reference). In this regard, the compositions may be delivered subcutaneously, epidermally, intradermally, intrathecally, intraorbitally, intramucosally, intraperitoneally, intravenously, intraarterially, orally, intrahepatically or intramuscularly. Other modes of administration include oral and pulmonary administration, suppositories, and transdermal applications. In particularly preferred embodiments, the compositions may administered intravenously in the portal vasculature or hepatic artery. A clinician specializing in the treatment of patients with disorders of the blood (e.g., hemophilia) may determine the optimal route for administration of the chimeric AAV vectors comprising hFIX nucleic acid sequences based on a number of criteria, including, but not limited to: the serotype(s) of the chimeric vector used, the condition of the patient, and the purpose of the treatment (e.g., treatment of or prophylaxis against hemorrhage, or surgical hemostasis).

One skilled in the art will recognize that the methods and compositions described above are also applicable to a range of other treatment regimens known in the art. For example, the methods and compositions of the present invention are compatible with ex vivo therapy (e.g., where cells are removed from the body, incubated with the chimeric AAV particles and the treated cells are returned to the body).

Chimeric AAV particles may be administered to any tissue suitable for expression of proteins or fragments thereof. In a preferred embodiment, the chimeric AAV particles of the present invention (e.g., chimeric AAV particles comprising human Factor IX or human α1-antitrypsin) may be administered via the portal vasculature or hepatic artery where it is thought, without being bound by theory, that the vector transduces hepatocytes. Current approaches to targeting genes to the liver have focused upon ex vivo gene therapy. Ex vivo liver-directed gene therapy involves the surgical removal of liver cells, transduction of the liver cells in vitro (e.g., infection of the explanted cells with recombinant retroviral vectors) followed by injection of the genetically modified liver cells into the liver or spleen of the patient. A serious drawback for ex vivo gene therapy of the liver involves the lack of success in maintaining and expanding hepatocytes in culture. The success of ex vivo liver-directed gene therapy, therefore, depends upon the ability to efficiently and stably engraft the genetically modified (i.e., transduced) hepatocytes and their progeny. It has been reported that even under optimal conditions, autologous modified liver cells injected into the liver or spleen that successfully engraft represent only a small percentage (less than 10%) of the total number of cells in the liver. Ectopic engraftment of transduced primary hepatocytes into the peritoneal cavity has also been attempted, in order to address the problem of engraftment in the liver.

Given the problems associated with ex vivo liver-directed gene therapy, in vivo approaches have been investigated for the transfer of genes into hepatocytes, including the use of recombinant retroviruses, recombinant adenoviruses, liposomes and molecular conjugates. While these in vivo approaches do not suffer from the drawbacks associated with ex vivo liver-directed gene therapy, they do not provide a means to specifically target hepatocytes. In addition, several of these approaches require performance of a partial hepatectomy, in order to achieve prolonged expression of the transferred genes in vivo. The present invention provides compositions and methods for the long-term expression of biologically active Factor IX or human α1-antitrypsin. It is contemplated that the present invention will bypass the need for partial hepatectomy, while allowing expression of Factor IX or human α1-antitrypsin in concentrations that are therapeutic in vivo. The present invention further provides compositions and methods useful for gene therapy that target hepatocytes for the production of Factor IX or human α1-antitrypsin in treated individuals.

Cells derived from other tissues are also suitable for the expression of Factor IX or human α1-antitrypsin. Muscle cells, for example, have been shown to express biologically active blood clotting Factor IX which is normally synthesized in the liver.

Finally, the chimeric AAV particles may contain any nucleic acid sequences coding for biologically active full length Factor IX or human α1-antitrypsin. Alternatively, the chimeric AAV vectors may contain nucleic acid sequences encoding a functional fragment of Factor IX or human α1-antitrypsin which retains Factor IX or human α1-antitrypsin biological activity. Administration of chimeric AAV particles comprising Factor IX, or human α1-antitrypsin, or a functional fragment thereof to a patient can restore Factor IX activity such that the blood clotting time in the patient is reduced, or human α1-antitrypsin activity such that serine proteinase inhibition is resumed.

Delivery of hFIX via the chimeric vectors of the present invention provides a significant improvement over the previously available therapeutic regimens for the treatment of hemophilia patients. Because administration of chimeric AAV vectors comprising hFIX dramatically reduces the risk of acquiring diseases associated with blood-borne pathogens, it provides a much safer alternative therapy to that of administration of pdFIX. Moreover, delivery of hFIX via chimeric vectors provides a more efficient method for maintenance of therapeutically effective levels of circulating hFIX than repeated injections of either pdFIX or rFIX because it may provide complete and persistent correction of the hemophilia phenotype.

The following examples are provided to illustrate certain embodiments of the invention. They are not intended to limit the invention in any way.

### EXAMPLE I

Production and Purification of Chimeric AAV Vectors Derived from AAV1 and AAV2

Novel chimeric rAAV particles of the present invention comprise both AAV1 and AAV2 capsid proteins and were generated utilizing a mixture of AAV1 and AAV2 helper constructs. The AAV1 helper construct included the AAV2 rep gene and AAV1 cap gene and the AAV2 helper construct included the AAV2 rep gene and the AAV2 cap gene. Chimeric rAAV vectors were generated using a four-component system comprised of a vector construct, an adenovirus helper virus plasmid, and different combinations of AAV1 and AAV2 helper constructs.

Materials and methods

Vector production and titering: Human 293 cells were maintained in Dulbecco's modified Eagle Medium (DMEM) supplemented with 10% fetal bovine serum (FBS), 1× Penicillin and Streptomycin and incubated in a humidified environment of 5% CO₂ at 37° C. Recombinant AAV vectors were produced as described previously (Xiao et al. 1998. J Virol 72: 1022-1026) The vector plasmids pAAV-CMV-FIX, pAAV1-EP-FIX, and pAAV-MFG-a1at were described elsewhere. (Herzog et al. 1997, Proc. Natl. Acad. Sci. USA, 94:5804-9.) Briefly, helper plasmid, vector plasmid and mini adenovirus helper plasmid were transfected to 293 cells at a ratio of 1:1:2 by Calcium Phosphate precipitation. The transfected cells were then maintained in DMEM media containing 2% FBS. At four days post-transfection, the cells were harvested and vectors were purified by CsCl gradient or over heparin columns. Vectors were titered by quantitative PCR using ABI Biosystems 7700 as previously described (Cao et al., 2000, J Virol 74:11456-11463). Viral titers were confirmed by Slot blot analysis.

Vector Purification by CsCl Gradient:

Cell pellets were resuspended in 10 mM Tris (pH 8.0) and sonicated to disrupt the cell membranes. After incubating with DNAse and RNAse, 1/20 volume of 10% sodium deoxycholate solution was added to the mix to complete the lysis process. Cell lysates were subjected to two rounds of centrifugation in an ultracentrifuge, wherein the first round was performed at 38,000 rpm, 4° C., for 24 hours (Sw28 rotor) and the second round was performed at 16,000 rpm, at 4° C., for 24 hours (Ti70 rotor). The fractions with a refractory index in the range of 1.370-1.377 in the first round were collected and used for the 2^{nd} round of centrifugation. The resulting peak fractions were then collected, pooled together and dialyzed against phosphate buffered saline (PBS) containing 10% glycerol.

Vector Purification by Heparin Column:

Cell pellets were resuspended in 10 mM Tris (pH 8.0) +150 mM sodium chloride and sonicated for 2 minutes to release the virus. The resulting mixture was centrifuged at 3000 g and the debris was discarded. The supernatant was incubated with Benzonase (Sigma) in the presence of 0.5% sodium deoxycholate. Heparin sepharose resin was added to the mixture prior to incubation overnight at room temperature with agitation. Vectors were eluted in different 10 mM Tris (pH 8.0) solutions containing a gradient of salt concentrations ranging from 200 mM NaCl to 1000 mM NaCl.

Animal Handling and Vector Administration:

The C57BL/6 (Charles River Breeding Laboratories) mouse strain was selected for intramuscular injection with rAAV. CD4 knock out (KO) mice were purchased from The Jackson Laboratory, Bar Harbor, Me. The animals were 4-6 weeks old when vectors were administered. Standard procedures for animal handling were followed. AAV-FIX was injected into the tibialis anterior (25 μl) and the quadriceps muscle (50 μl) of the left leg using a Hamilton syringe to target muscle. To target liver, vectors were injected through the tail vein. Blood samples were collected every two weeks from the retro-orbital plexus in microhematocrit capillary tubes and the plasma assayed for hFIX expression by ELISA (vide infra). A typical group usually consisted of three mice. Comparable methods were used for administration of pAAV-MFG-a1at to mice.

In vitro Neutralization Assay:

For neutralization assays, 293 cells were infected with vectors at a multiplicity of infection (MOI) of 5000. To test whether the vector can be neutralized by AAV1 and AAV2 antiserum, the vectors were incubated with AAV antiserum at 1:100 dilution for 30 min at 37° C. The “neutralized” vectors and controls were subsequently used to infect 293 cells. At 48 hours post infection, the media was harvested and tested for the presence of secreted human Factor IX by ELISA.

Factor IX (FIX) ELISA:

Human FIX antigen in mouse plasma was determined by ELISA as described by Walter and High (1997, Adv. Vet. Med. 40:119-134), which can distinguish human FIX from mouse FIX. All samples were measured in duplicate. The dilution of mouse serum was usually in the range of 5-50 fold depending on the level of human Factor IX in the serum or media.

Human α1-Antitrypsin ELISA:

The concentration of human α1-antitrypsin in the mouse serum was measured by ELISA. Briefly, 10 μg/ml rabbit anti-human α1-antitrypsin (Sigma) was coated to ELISA plates in 0.1 M/sodium bicarbonate, pH 9.6, for 2 hours. After being blocked with 3% BSA/PBS, different dilutions (50 to 200 times dilution) of mouse serum samples were applied. The captured human α1-antitrypsin was incubated with goat anti-human α1-antitrypsin (2 μg/ml; Sigma) followed by mouse anti-goat IgG peroxidase conjugate (Sigma). Signal detection was achieved by addition of ABTS (Boehringer Mannheim). The linear range of this assay is between 0.3 and 30 ng/ml. The human α1-antitrypsin standard was purchased from The Scripps Institute (La Jolla, Calif.).

Results

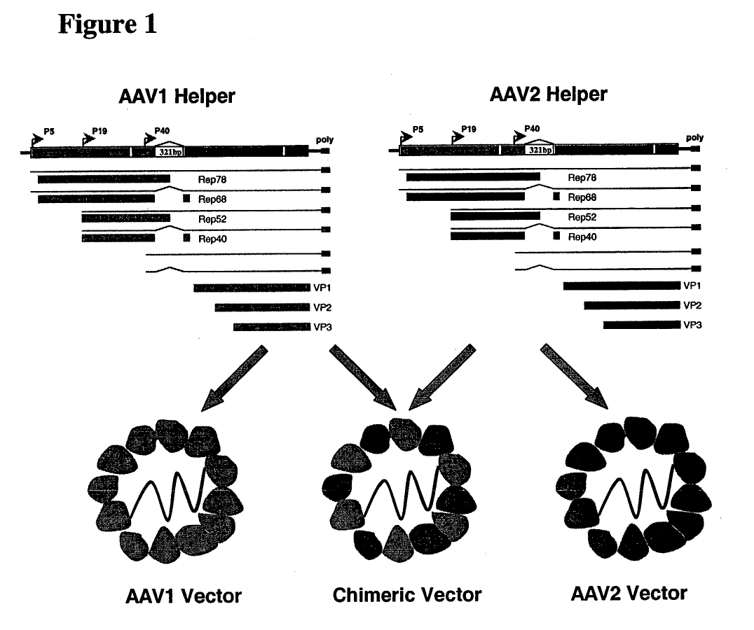

FIG. 1 illustrates the novel strategy for the generation of the chimeric AAV vectors of the present invention.

TABLE II
shows the ratios of AAV1/AAV2 used in the helper
plasmid mix and the yield of viral vectors produced
following infection with a particular mix. Ratios are
indicated in parentheses.

| Virus | Final | AAV1:AAV2 | | |
|---|---|---|---|---|
| # | vector | Helper ratio (μg) | % AAV1 | % AAV2 |
| 1 | AAV1 | 50:0 | 100 | 0 |
| 2 | AAV1/2 (9:1) | 45:5 | 90 | 10 |
| 3 | AAV1/2 (1:1) | 25:25 | 50 | 50 |
| 4 | AAV1/2 (1:9) | 5:45 | 10 | 90 |
| 5 | AAV2 | 0:50 | 0 | 100 |

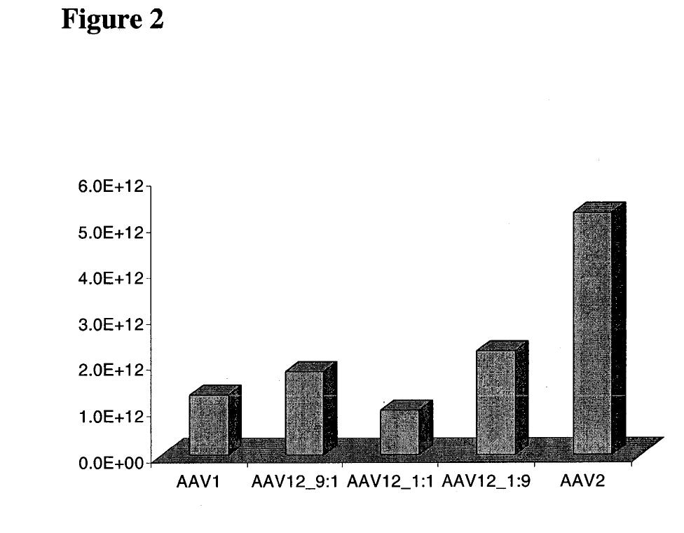

Table II shows the ratios of different combinations of AAV1 and AAV2 helper constructs which were used to produce chimeric rAAV vectors. In brief, vector construct, adenovirus helper plasmid, and AAV1/AAV2 helper plasmid mix were cotransfected into human 293 cells. Cells were harvested at 96 hours post-transfection and the cell pellets were collected and divided in half for differential processing. One half was used to purify the vector over a standard CsCl gradient. The other half was purified over a heparin column. The CsCl gradient was utilized to isolate packaged virions having a density between 1.38-1.42 g/ml. FIG. 2 shows the number and viral capsid composition of packaged virions purified by the above means. In accordance with previous results, the yield of AAV1 vector was 3-4 fold lower than that of AAV2. The chimeric vector yield was in the ranges normally observed for AAV1 and AAV2, depending on the composition of the helper plasmids used in the helper plasmid mix. These data suggested that the mixing of helper plasmids did not interfere with AAV packaging.

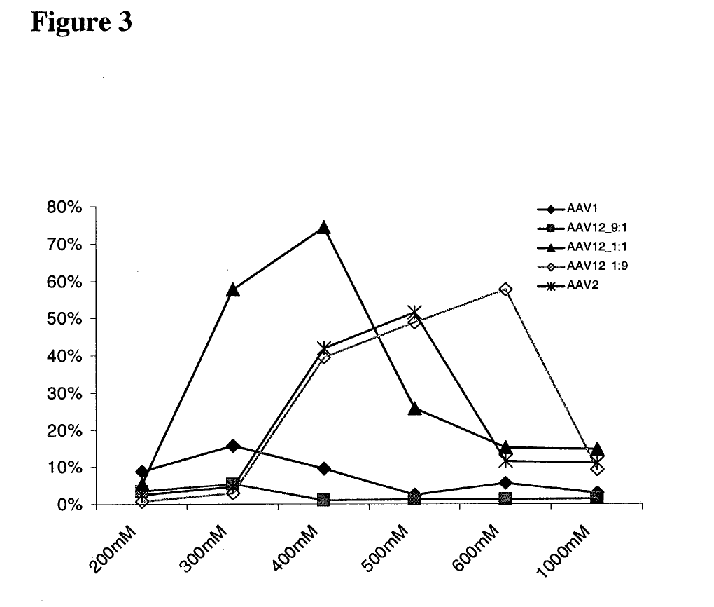

Heparin sulfate proteoglycan has been identified as an AAV2 receptor. The strong affinity of AAV2 for heparin facilitates purification of AAV2 particles by chromatography over heparin columns. AAV2 vectors purified by this method can be of high purity. Since AAV1 utilizes different cellular receptors, AAV1 is thought to exhibit limited or no affinity for heparin. Accordingly, chimeric AAV vectors were evaluated based on their ability to bind heparin columns. FIG. 3 shows the amount of viral vector in each elution fraction. The Y-axis represents the total amount of vector in each fraction and the X-axis represented the salt concentration used for each elution. As shown in FIG. 3, AAV1 particles did not bind to heparin columns and thus provided a negative control for this assay. In contrast, the chimeric vectors displayed affinity for heparin, a property specific for AAV2. Of note, the elution peak of chimeric vectors from heparin columns was shifted to progressively lower concentrations of salt when the ratio of AAV2 helper was reduced to less than 50%. This phenomenon reflected a shift toward features more typical of particles derived from infection with AAV1 helper plasmid.

Characterization of Antigenic Properties of Chimeric AAV Vectors in vitro.

Antigenic properties of chimeric AAV1/AAV2 vectors were characterized in vitro. Briefly, COS cells were plated at 24 hours before infection with rAAV vectors at a multiplicity of infection (MOI) of 5000 particles per cell. The following vectors, AAV1, AAV2, and chimeric AAV1/AAV2 (produced following cotransfection with AAV1 and AAV2 at ratios of 9:1, 1:1 and 1:9; as shown in FIG. 2) were incubated with AAV1 and AAV2 neutralizing antibodies (NAbs) at a dilution of 1:100 or heparin at 100 μg/ml. Control groups were incubated with mouse serum at a dilution of 1:100. The levels of the transgene secreted Factor IX were measured 48 hours post infection by ELISA (Table III).

| | | AAV1/2 | AAV1/2 | AAV1/2 | |
|---|---|---|---|---|---|
| | AAV1 | (9:1) | (1:1) | (1:9) | AAV2 |
| AAV1- | 3% | 1.80% | 10% | 100% | 100% |
| Antiserum | | | | | |
| AAV2- | 78% | 52% | 3.60% | 0% | 0% |
| Antiserum | | | | | |

The data indicate that the chimeric vectors could be neutralized by either AAV1 or AAV2 NAbs, which demonstrated that the chimeric AAV1/AAV2 viroids had acquired properties distinct to both AAV1 and AAV2. Since AAV2 neutralizing antibodies cross-react with AAV1 and partially reduce its ability to infect cells, further inhibition studies were performed. Heparin, for example, is known to neutralize only those viral vectors which express AAV2 capsid proteins on their surface. Accordingly, heparin was also tested to evaluate its inhibitory effects on AAV1/AAV2 chimeric vector infectivity. Pre-incubation of AAV1/AAV2 chimeric vector with heparin did neutralize the ability of AAV1/AAV2 chimeric vector to infect cells (data not shown). In summary, the data confirmed that the methods of the present invention can be used to produce a near uniform vector which has both AAV1 and AAV2 capsid proteins, in accordance with the ratio of helper plasmids used in the initial co-transfection.

Characterization of the Performance of Chimeric AAV1/2 Vectors in vivo.

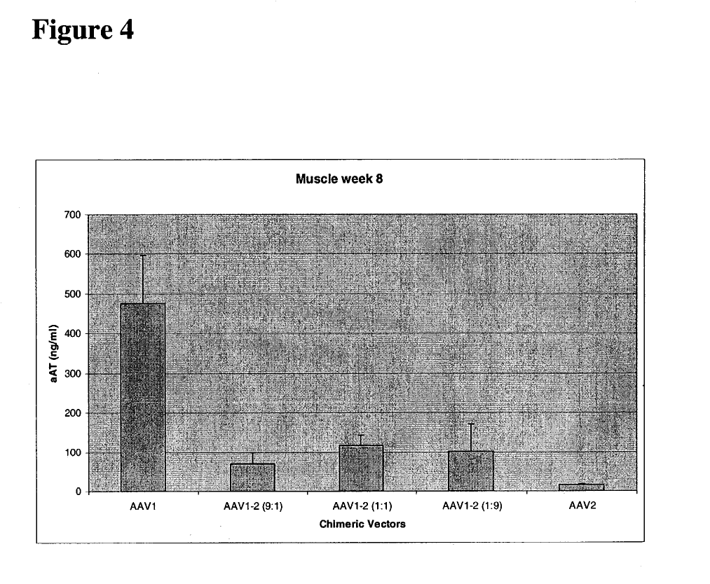

To evaluate the ability of these chimeric AAV1/AAV2 vectors to infect cells in vivo (FIG. 4), wild type and chimeric AAV vectors were injected into the muscle of CD4 knock out (KO) mice. The CD4 KO mouse strain was chosen for these experiments because it is impaired immunologically and, therefore, fails to generate NAbs against the transgene human Factor IX which is expressed following productive transduction of viroids. Approximately 5×10¹⁰ vector particles including CsCl purified AAV1, AAV2, and chimeric AAV1/AAV2 particles (produced following cotransfection with AAV1 and AAV2 at ratios of 9:1, 1:1 and 1:9) were injected intramuscularly into CD4 KO mice. The expression of human Factor IX was assayed by ELISA at week eight post-injection. FIG. 4 shows the amount of human Factor IX detected in the sera of mice injected with the different AAV vectors. The AAV1/AAV2 chimeric vectors demonstrated an improved ability to infect muscle cells relative to that of wild type AAV2 as indicated by increased levels of circulating human Factor IX.

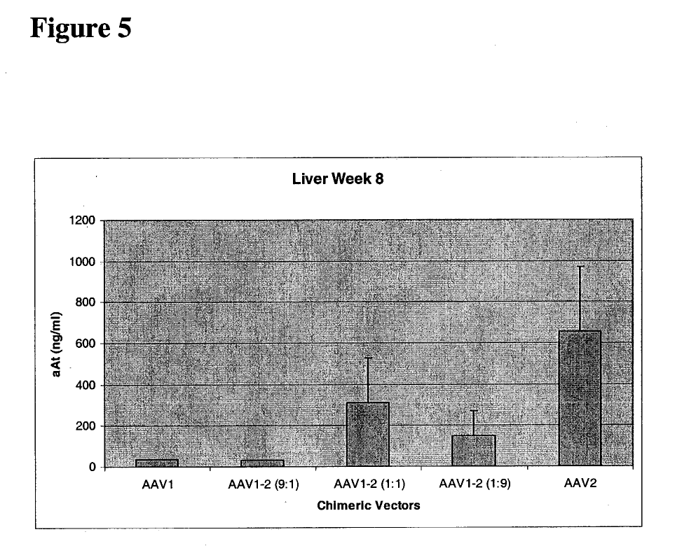

As a comparison, 5×10¹ AAV1, AAV2, and chimeric AAV1/AAV2 particles (produced following cotransfection with AAV1 and AAV2 at ratios of 9:1, 1:1 and 1:9) were administered to the liver of C57BL6 mice. To target the liver, vectors were injected into the tail vein. The expression of human Factor IX was assayed by ELISA at week eight post-infection. FIG. 5 shows the peak levels of human Factor IX detected in sera from mice into which the different AAV vectors were injected. The amount of human Factor IX produced following infection with chimeric vectors, as detected by ELISA, generally exceeded that of the wild type AAV1 parental vector. The data shown in FIGS. 4 and 5 revealed that the chimeric vectors can infect both liver and muscle cells, thus demonstrating that the AAV1/AAV2 chimeric vectors of the present invention provide novel and improved tools for use in gene transfer experiments and therapy.

In further experiments, human factor IX or human α1-antitrypsin were used as the secretion marker for the study. Once again, CD4 KO mice were used to test the performance of vectors in muscle, and C57BL6 mice were used to test the performance of vectors in liver.

The experiments are summarized in Table IV, which shows relative expression levels of FIX and α-1AT in muscle and liver. Since these experiments were repeated using different batches of vectors and the absolute expression varies, the performance of AAV1 in liver and AAV2 in muscle is defined as 1, and the expression from other vectors is shown as “fold” compared to the standard, “1”.

| | Liver | Liver | Muscle | Muscle |
|---|---|---|---|---|
| | α-1AT | hFIX | α-1AT | hFIX |
| AAV1 | 1 | 1 | 4.6-6.3 | 5.4-8.9 |
| AAV12_9:1 | 1.6-10.3 | 1.4-5.4 | 2-3.4 | 1.8-3.1 |
| AAV12_1:1 | 3.8-17.4 | 6.6-23.7 | 8.5-11.7 | 2.3-7.1 |
| AAV12_1:9 | 2.4-13.5 | 5.3-22.7 | 1.4-2.9 | 1.6-3.0 |
| AAV2 | 2.1-5.7 | 2.7-12.2 | 1 | 1 |

The chimeric vectors exhibited improved performance over AAV2 in muscle. Although the AAV12_{—}1:1 vector consists of only 50% AAV1 capsid proteins, expression levels were nearly the same as those of AAV1, which is approximately 4- to 7-fold higher than AAV2. Further, in the liver, transduction with chimeric vectors containing at least 50% AAV2 capsid protein resulted in higher levels of transgene expression. Surprisingly, the expression of human factor IX or human α1-antitrypsin from chimeric vector improved to match the best performance of their parental vectors in the liver, which exhibited approximately 10-fold increases over that of AAV1. The data indicate that the chimeric vectors of the invention combine useful features of both AAV1 and AAV2.

### EXAMPLE II

Chimeric AAV Vectors are useful for the Therapeutic Treatment of Patients having a Variety of Disorders

The chimeric AAV vectors of the present invention may be used to treat a variety of diseases and disorders. In particular embodiments, the chimeric vectors of the present invention may be used to treat a patient with a disease characterized by a genetic and/or biochemical defect. A nucleic acid sequence encoding virtually any polypeptide or functional fragment thereof may be incorporated into the chimeric AAV vectors of the present invention to provide means to express the desired protein in a cellular context. One of skill in the art would appreciate that certain constraints are involved regarding the size of the nucleic acid sequence inserted into an AAV vector. A skilled artisan would, however, be able to truncate and/or modify a nucleic acid sequence encoding a polypeptide of interest so as to render it compatible with the packaging machinery of the vector system.

Table V provides exemplary nucleic acid sequences which may be incorporated into chimeric AAV vectors. All nucleic acid sequences are human in origin unless otherwise specified.

| | | |
|---|---|---|
| Disease | | |
| Accession # | Protein Deficiency | GenBank |

Autosomal Recessive Disorders

| | | |
|---|---|---|
| Adenosine | Adenosine deaminase | BC013329 |
| deaminase (ADA) | | |
| deficiency | | |
| Cystic Fibrosis | CF transmembrane | Human M28668 |
| (CF) | conductance regulator | Mouse M69298 |
| | (CFTR) | |
| Galactosemia, | Galctosemia-1- | NM_000155 |
| classic | phosphate-uridyl | |
| | transferase (GALT) | |
| Phenylketonuria | phenylalanine | K03020 |
| (PKU) | hydroxylase | |
| Sickle cell anemia | Hemoglobin | NM_000518 |
| and | | |
| Thalassema | | |
| Tay-Sachs | Hexosaminidase A | BC018927 |
| disease | | |
| Alpha-1-anti- | Alpha-1-anti- | V00496; K01396 |
| trypsin | trypsin | |
| deficiency | | |
| Canavan Disease | Asparoacylase | NM_000049 |
| Gaucher Disease | Glucocerebrosidase | D13286 |
| Niemann-Pick | Sphingomyelin | NM_000543 |
| Disease | phosphodiesterase 1 | |

X-linked Disorders

| | | |
|---|---|---|
| Hemophilia A | Factor VIII | BC022513 |
| Hemophilia B | Factor IX | J00136 |
| Lesch-Nyham | Hypoxanthifle guanine | NM_000194 |
| syndrome | phosphoribosyl | |
| | transferase (HPRT) | |

Autosomal Dominant Disorders

| | | |
|---|---|---|
| Familial hyper- | Low density | BC014960 |
| cholesterolemia | lipoprotein (LDL) | |
| | receptor | |
| Huntington's | HD protein | AK025918 |
| disease (HD) | | |
| Neurofibromatosis | NF1 protein | D42072 |
| (NF), type I | | |

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for making chimeric adeno-associated (AAV) particles comprising capsid proteins from different AAV serotypes, said method comprising:
a) providing a vector construct wherein said vector construct encodes nucleic acid sequences encoding a polypeptide;
b) providing a helper virus construct wherein said helper virus provides accessory functions for production of viral particles;
c) combining at least two AAV helper constructs to produce an AAV helper construct mixture;
d) introducing said vector construct, said helper virus construct, and said AAV helper construct mixture into a recipient cell to effect production of chimeric AAV particles capable of infecting diverse cell types.

2. A method as claimed in claim 1, wherein said AAV helper construct mixture comprises four different AAV helper constructs.

3. A chimeric AAV particle produced by the method of claim 1.

4. A method for making chimeric adeno-associated (AAV) particles comprising capsid proteins from two different AAV serotypes, said method comprising:
a) providing a vector construct wherein said vector construct encodes nucleic acid sequences encoding a polypeptide;
b) providing a helper virus construct wherein said helper virus provides accessory functions for production of viral particles;
c) selecting a first AAV helper construct comprising nucleic acids encoding a first capsid protein;
d) selecting a second AAV helper construct comprising nucleic acids encoding a second capsid protein;
e) combining said first and second AAV helper constructs to produce an AAV helper construct mixture;
f) introducing said vector construct, said helper virus construct, and said AAV helper construct mixture into a recipient cell to effect production of chimeric AAV particles capable of infecting diverse cell types.

5. A method as claimed in claim 4, wherein said AAV helper construct mixture comprises AAV1 and AAV2 helper constructs.

6. A method as claimed in claim 5, wherein said AAV helper construct mixture is comprised of a ratio of AAV1 to AAV2 helper constructs wherein said ratio is comprised of 20 percent AAV1 helper construct and 80 percent AAV2 helper construct.

7. A method as claimed in claim 5, wherein said AAV helper construct mixture is comprised of a ratio of AAV1 to AAV2 helper constructs wherein said ratio is comprised of 50 percent AAV1 helper construct and 50 percent AAV2 helper construct.

8. A method as claimed in claim 5, wherein said AAV helper construct mixture is comprised of a ratio of AAV1 to AAV2 helper constructs wherein said ratio is comprised of 80 percent AAV1 helper construct and 20 percent AAV2 helper construct.

9. A chimeric AAV particle produced by the method claim 4.

10. A chimeric AAV particle comprising capsid proteins from AAV1 and AAV2 having increased tissue tropism, said particle further comprising a sequence encoding a therapeutic protein of interest.

11. The chimeric AAV particle of claim 10, wherein said sequence encoding a therapeutic protein of interest is selected from the group consisting of SEQ ID NO: 1, SEQ ID NO: 2 or a sequence set forth in Table V.

12. The chimeric AAV particle as claimed in claim 11, wherein said nucleic acid sequence comprises SEQ ID NO: 1 which encodes Factor IX polypeptide.

13. The chimeric AAV particle as claimed in claim 11, wherein said nucleic acid sequence comprises SEQ ID NO: 2 which encodes human α1-antitrypsin polypeptide.

14. The method as claimed in claim 4, wherein said polypeptide is encoded by a nucleic acid sequence selected from the group of nucleic acid sequences provided in Table V.

15. A pharmaceutical composition comprising the chimeric AAV particle of claim 10 in a biocompatible pharmaceutical carrier.

16. A pharmaceutical composition comprising the chimeric AAV particle of claim 11 in a biocompatible pharmaceutical carrier.

17. A pharmaceutical composition comprising the chimeric AAV particle of claim 12 in a biocompatible pharmaceutical carrier.

18. A pharmaceutical composition comprising the chimeric AAV particle of claim 13 in a biocompatible pharmaceutical carrier.

19. A method of gene therapy for the treatment of a patient having a mutation in a gene, said method comprising delivery of a therapeutically effective amount of said composition of claim 15 to cells of said patient.

20. A method of gene therapy for the treatment of a patient having a Factor IX deficiency, said method comprising delivery of a therapeutically effective amount of said composition of claim 17 to cells of said patient.

21. A method of gene therapy for the treatment of a patient having a alpha 1-antitrypsin deficiency, said method comprising delivery of a therapeutically effective amount of said composition of claim 18 to cells of said patient.

22. The method of claim 20, wherein said cells are selected from the group consisting of muscle cells and, liver cells.

23. The method of claim 21, wherein said cells are selected from the group consisting of muscle cells and liver cells.
